(19) United States
(12) Patent Application Publication
(10) Pub. No.: US 2026/0115332 A1
(43) Pub. Date: Apr. 30, 2026

(54) Fibroblast Activation Protein Alpha-Cleavable Pro-Peptides and Methods of Use

(71) Applicant: The Regents of the University of California, Oakland, CA (US)

(72) Inventors: Michael J. Evans, San Francisco, CA (US); Charles S. Craik, San Francisco, CA (US)

(21) Appl. No.: 19/100,058

(22) PCT Filed: Aug. 25, 2023
(86) PCT No.: PCT/US2023/072939

§ 371 Date: Jan. 30, 2025

Related U.S. Application Data
(60) Provisional application No. 63/401,008, filed on Aug. 25, 2022.

(51) Int. Cl. A61K 51/08; A61P 35/00; C07K 14/46; C07K 14/705

(52) U.S. Cl. CPC A61K 51/088; A61P 35/00; C07K 14/463; C07K 14/705; A61K 2121/00; A61K 2123/00; C07K 2319/035; C07K 2319/50

(57) ABSTRACT

Provided are fibroblast activation protein alpha (FAPα)-cleavable pro-peptides that find use in treating conditions associated with FAPα expression. The FAPα-cleavable pro-peptides also find use in imaging locations of FAPα activity in vivo. In some embodiments, a pro-peptide of the present disclosure comprises a membrane interacting domain, a masking domain, and a FAPα cleavage site. The masking domain, when linked to the membrane interacting domain, is effective to inhibit interaction of the membrane interacting domain with a phospholipid bilayer. The FAPα cleavage site is disposed between the membrane interacting domain and the masking domain. The membrane interacting domain may be conjugated to one or more therapeutic agents, non-limiting examples of which include radioisotopes. Also provided are methods of treating a condition associated with FAPα expression in a subject in need thereof, such methods comprising administering an effective amount of a pro-peptide of the present disclosure to the subject.

### CROSS-REFERENCE TO RELATED APPLICATIONS

This application is a national stage filing under 35 U.S.C. 371 of International Patent Application No. PCT/US2023/072939, filed Aug. 25, 2023, which claims the benefit of U.S. Provisional Patent Application No. 63/401,008, filed Aug. 25, 2022, which application is incorporated herein by reference in its entirety.

### STATEMENT OF GOVERNMENT SUPPORT

This invention was made with government support under R01 CA258297, and R01 AI161027 awarded by the National Institutes of Health. The government has certain rights in the invention.

### INCORPORATION BY REFERENCE OF SEQUENCE LISTING

A Sequence Listing is provided herewith as a Sequence Listing XML, “UCSF-680WO_SEQ_LIST” created on Aug. 24, 2023 and having a size of 48,677 bytes. The contents of the Sequence Listing XML are incorporated by reference herein in their entirety.

### INTRODUCTION

Recent FDA approvals for lutetium Lu 177 dotatate (Lutathera®), iobenguane I 131 (Azedra®) and ¹⁷⁷Lu-PSMA 617 (Pluvicto®), in conjunction with the emergence of promising experimental agents in clinical trials underscore the surging enthusiasm to investigate targeted radiotherapy (TRT) as a treatment modality for cancers. However, the clinical experience with TRTs shows that they are rarely curative, and tumor responses are typically transient and/or variable among patients. Thus, new strategies to maximize the therapeutic benefit of TRT for cancer patients are needed.

Within the past 10 years, the nuclear medicine field has prioritized the development of low MW radioligand therapies (RLTs) that rapidly exit the bloodstream to minimize host toxicity yet are still effective antitumor agents by targeting highly overexpressed cancer proteins. This transition was motivated by 30 years of clinical experiences with various high MW radioligands like immunoglobulins, which showed that their long serum half-life (3-7 days) results in radiation exposure to sensitive normal tissue compartments (e.g., bone marrow) that narrows or eliminates a therapeutic index. The evolution of radioligands targeting prostate specific membrane antigen (PSMA) stands out as an instructive case study on the impact of radioligand pharmacokinetics (PK) on therapeutic index. While various radiolabeled forms of the IgG J591, including ¹⁷⁷Lu-J591, stalled in clinical trials due to dose limiting toxicities, Pluvicto® (¹⁷⁷Lu-PSMA 617) a low MW radioligand with weaker affinity for PSMA and lower tumor uptake compared to J591, nevertheless achieved FDA approval for prostate cancer treatment in 2022 due in large part to its better safety profile.

However, treating cancer with low MW RLTs is challenging for a number of reasons. First, to deliver a sufficient dose to tumors, the field is limited to the small minority of highly overexpressed proteins in cancer that can extract sufficient RLT from circulation as the drug is rapidly exiting the body. Indeed, prominent RLT drug targets like PSMA, somatostatin receptor type 2, carbonic anhydrase 9, and the bombesin receptor are all very highly overexpressed on cancer cells (at least 105 receptors per cell). Second, ligand/receptor complexes are intrinsically unstable in biology, and subject to dissociation or degradation after endocytosis. Indeed, longitudinal PET studies in patients have shown that RLTs typically clear from tumors within 96 hours, and in some extreme cases (e.g., FAPI PET) the radioisotope can depart the tumor within a few hours.

Fibroblast Activation Protein-α (FAPα) is a type II integral serine protease expressed by activated fibroblasts. Cancer-associated fibroblasts (CAFs) in the tumor stroma exhibit abundant and stable expression of FAPα, which plays an important role in promoting tumor growth, invasion, metastasis, and immunosuppression. The protein encoded by the human FAPα gene is a 760 amino acid single pass type II transmembrane protein composed of a short cytoplasmic N terminal part (6 amino acids), a transmembrane region (amino acids 7-26), and a large extracellular domain. FAP is enzymatically active as a homodimer. It exhibits both a post-proline dipeptidyl peptidase and endopeptidase activity, both of which are dependent on the catalytic triad comprising Ser624 Asp702 His734 in human and mouse FAPα. Due to the unique structure of proline, most proteases do not cleave the peptide bonds adjacent to it. In several cases, the presence of proline thus acts as a mechanism that prevents protein degradation or cleavage.

Normal tissues have low and generally undetectable levels of FAPα expression. However, FAPα is overexpressed in many tumor tissues, including breast, colorectal, pancreatic, lung, brain, intrahepatic bile duct, and ovarian cancers. In addition, high levels of FAPα expression can be detected in some tumors that are derived from non-epithelial tissues, such as melanoma and myeloma. In these tumors, FAPα overexpression is often observed in the interstitium, which has led to FAPα being considered a universal marker for CAFs, although FAPα expression can also be detected in gastric carcinoma, pancreatic carcinoma and melanoma cells. Details regarding FAPα expression in human malignancies can be found, e.g., in Busek et al. (2018) Frontiers In Bioscience, Landmark 23:1933-1968.

### SUMMARY

Provided are fibroblast activation protein alpha (FAPα)-cleavable pro-peptides that find use in treating conditions associated with FAPα expression. The FAPα-cleavable pro-peptides also find use in imaging locations of FAPα expression in vivo. In some embodiments, a pro-peptide of the present disclosure comprises a membrane interacting domain, a masking domain, and a FAPα cleavage site. The masking domain, when linked to the membrane interacting domain, is effective to inhibit interaction of the membrane interacting domain with a phospholipid bilayer. The FAPα cleavage site is disposed between the membrane interacting domain and the masking domain. The membrane interacting domain may be conjugated to one or more therapeutic agents, non-limiting examples of which include radioisotopes. Also provided are methods of treating a condition associated with FAPα expression in a subject in need thereof, such methods comprising administering an effective amount of a pro-peptide of the present disclosure to the subject.

### BRIEF DESCRIPTION OF THE FIGURES

FIG. 1: Top: A schematic illustration of a pro-peptide (sometimes referred to herein as a “restricted interaction peptide” or “RIP”) according to embodiments of the present disclosure. The RIPs are low MW (˜4 kDa) peptides comprising three domains from N-terminal to C-terminal: (i) a membrane interacting domain (in this example, a membrane binding antimicrobial peptide (AMP)) coupled to a payload (e.g., a chelator bound to a radioisotope); (ii) an endoprotease cleavage site, e.g., spanning P4-P4′; and (iii) a peptide “masking domain” that prevents the AMP from adopting a helical conformation required for membrane binding. Bottom: In this example, upon cleavage of the pro-peptide by the target endoprotease in vivo, the radiolabeled AMP is liberated, spontaneously adopts a helical conformation and attaches to a nearby phospholipid membrane.

FIG. 2: A schema showing the proposed advantage of RIP-based targeted radiotherapy (TRT). At left is shown the typical blood and tumor activity curve for large molecular weight targeted radiotherapies (e.g., immunoglobulins). High MW targeted radiotherapies have high tumor uptake but dose limiting toxicity due to slow clearance from blood. Low MW targeted radiotherapies (e.g., small molecule radioligand therapies) benefit from improved safety due to rapid serum clearance but rapid clearance by nature limits tumor exposure. RIPs achieve both desirable safety due to rapid clearance and high tumoral AUC due to the renewable, catalytic amplification of isotope accumulation in tumors as well as the persistent binding of membrane interacting peptides to cell membranes.

FIG. 3: Discovery of unique candidate P4-P4′ fibroblast activation protein alpha (FAPα) substrate sequences via Multisubstrate profiling with Mass Spectrometry (MSP-MS). Top: an Icelogo representing the cumulative amino acid preferences for recombinant human FAPα. Bottom: a heat map summarizing the amino acids that were most frequently incorporated at each site in the cleavage products. Lighter indicates an enriched amino acid. X=no preferred amino acid.

FIG. 4A-4C: Kinetic data for three of the top P4-P4′ 8 mer peptides emerging from an MSP-MS assay. The kinetic properties were determined using FRET donor/acceptor substrate adducts and human recombinant FAP alpha.

FIG. 5: A schematic illustration of a pro-peptide (SEQ ID NO:53) according to embodiments of the present disclosure. From N- to C-terminus in this non-limiting example (sometimes referred to herein as “FRIP2”): (1) a ⁶⁴Cu labeled membrane interacting/binding domain; (2) a FAPα cleavage site; and (3) a masking domain.

FIG. 6A-6B: Representative coronal PET/CT images from male nu/nu mice bearing subcutaneous U87 tumors show that ⁶⁴Cu-FRIP2 has higher tumoral uptake compared to FRIP1, FRIP3, and two negative control RIPs, PAR1 (cut by thrombin) and GRIP B (cut by granzyme B). The images were acquired at 24 hours post injection. At right is shown the tumoral SUVmean values for each mouse cohort. *P<0.01.

FIG. 7: Assessment of ⁶⁴Cu-FRIP2 uptake in U87 tumors compared to tumor models lacking FAPα. The data demonstrates specificity of FRIP2 for FAPα.

FIG. 8: Biodistribution data collected 24 hours post injection of ⁶⁴Cu-FRIP2 in male nu/nu mice bearing subcutaneous U87 MG xenografts show the high uptake in the tumor compared to normal tissues. The normal tissues with the highest uptake were liver and kidney.

FIG. 9A-9B: Data demonstrating superior tumoral uptake of a labeled pro-peptide of the present disclosure (⁶⁴Cu-FRIP2 in this example) as compared to FAPI-46.

FIG. 10A-10B: Longitudinal PET/CT studies showing the uptake of ⁶⁴Cu-FRIP2 and ⁶⁴Cu-FAPI 46 in PC3-PIP tumors, a model with low FAP alpha expression. ROI analysis (FIG. 10A) of tumoral uptake shows that ⁶⁴Cu-FRIP2 was significantly higher in tumors compared to ⁶⁴Cu-FAPI 46. Shown in FIG. 10B are representative transverse images showing the tumoral uptake of either radiotracer over time. The orange arrow indicates the position of the tumor.

FIG. 11: A schematic illustrating the advantages of the RIP-based approach for targeted radiotherapy (TRT).

FIG. 12A-12C: ⁶⁷Cu-FRIP2 anti-tumor assessment data. FIG. 12A: Relative changes in tumor volume from cohorts of male nu/nu mice bearing subcutaneous U87 xenografts treated with either vehicle or ⁶⁷Cu-FRIP2. Mice (n=8/arm) were treated with a single IV bolus of ⁶⁷Cu-FRIP2 (1 mCi/mouse) on day 0 of the study. FIG. 12B-12C: IHC images of FAP alpha staining of a U87 xenograft. Staining of a HEK-FAP tumor (HEK293 stably overexpressing human FAP alpha) is shown to contextualize the level of staining in U87.

FIG. 13A-13B: Additional ⁶⁷Cu-FRIP2 anti-tumor assessment data. FIG. 13A: Survival curves for each cohort. The endpoints were (1) tumor volume exceeding 2000 mm³ or (2) body weight loss exceeding 20%. FIG. 13B: Relative changes in body weight for the mice in the cohort.

FIG. 14: A spider plot showing the individual tumor volume changes for the mice represented.

FIG. 15A-15B: A spider plot (FIG. 15A) of tumor volumes from an antitumor assessment study in male nu/nu mice bearing subcutaneous U87 tumors treated with vehicle, ⁶⁷Cu-FAPI 46, or ⁶⁷Cu-FRIP2. The mice received 1 mCi of radioactivity in a single IV administration on day 0 of the study. FIG. 15B: Relative tumor volumes from each cohort (n=8 mice/arm) on day 13, the latest date on which all mice in the study were still viable. ⁶⁷Cu-FRIP2 more potently suppresses tumor growth than ⁶⁷Cu-FAPI 46. *P<0.01.

FIG. 16: Survival curve for each cohort shown in FIG. 15. The endpoints were (1) tumor volume exceeding 2000 mm³ or (2) body weight loss exceeding 20%.

FIG. 17A-17B: Radioligand therapy with ⁶⁷Cu-FRIP2 in PC3-PIP. FIG. 17A: Day 69 antitumor assessment data showing the impact of ⁶⁷Cu-FRIP2 treatment on PC3-PIP subcutaneous xenografts (P<0.01). Mice were treated with vehicle (N=6) or 1mCi ⁶⁷Cu-FRIP2 (N=9) on day 0. FIG. 17B: Survival data plotted on a Kaplan-Meier curve shows significantly extended survival (P<0.001) of the treated arm compared to the arm that received the vehicle.

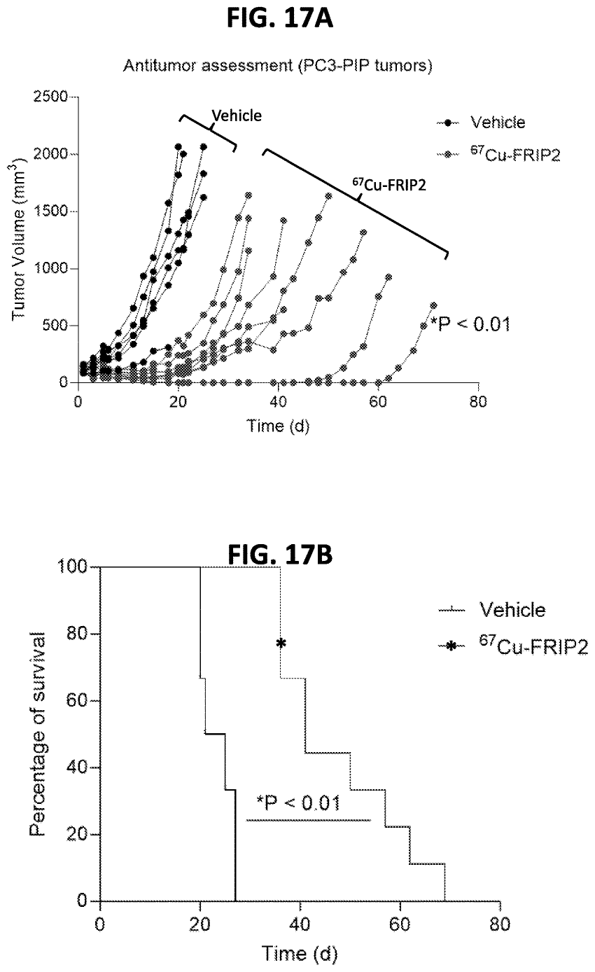

FIG. 18: Mouse weight changes in two cohorts treated with vehicle or 1mCi ⁶⁷Cu-FRIP2 on day 0. No unsafe weight changes were observed in the treated arms.

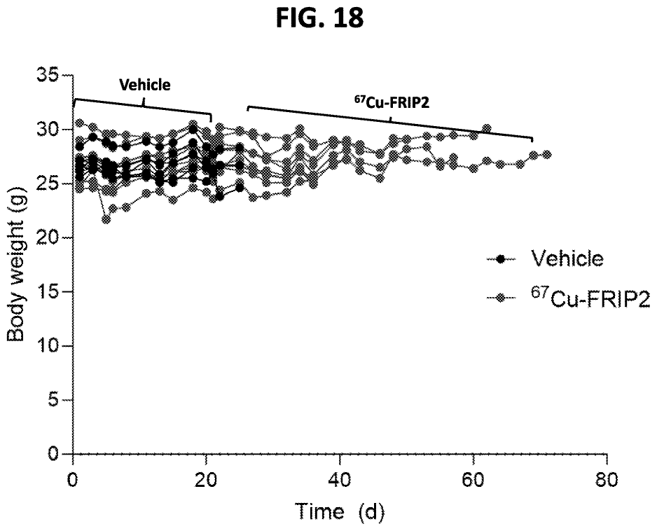

FIG. 19A-19B: Radioligand therapy with ⁶⁴Cu-FRIP2 in U87 MG. FIG. 19A: Day 27 antitumor assessment data showing the impact of ⁶⁴Cu-FRIP2 treatment on U87 MG subcutaneous xenografts (P<0.01). Mice were treated with vehicle (N=8) or 1.5 mCi ⁶⁴Cu-FRIP2 (N=8) on day 0 and day 8. FIG. 19B: Survival data plotted on a Kaplan-Meier curve shows significantly extended survival (P<0.01) of the treated arm compared to the arm that received the vehicle.

### DETAILED DESCRIPTION

Before the pro-peptides and methods of the present disclosure are described in greater detail, it is to be understood that the pro-peptides and methods are not limited to particular embodiments described, as such may, of course, vary. It is also to be understood that the terminology used herein is for the purpose of describing particular embodiments only, and is not intended to be limiting, since the scope of the pro-peptides and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the pro-peptides and methods. The upper and lower limits of these smaller ranges may independently be included in the smaller ranges and are also encompassed within the pro-peptides and methods, subject to any specifically excluded limit in the stated range. Where the stated range includes one or both of the limits, ranges excluding either or both of those included limits are also included in the pro-peptides and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the pro-peptides and methods belong. Although any pro-peptides and methods similar or equivalent to those described herein can also be used in the practice or testing of the pro-peptides and methods, representative illustrative pro-peptides and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present pro-peptides and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the pro-peptides and methods, which are, for clarity, described in the context of separate embodiments, may also be provided in combination in a single embodiment. Conversely, various features of the pro-peptides and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present pro-peptides and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Pro-Peptides

The present disclosure provides FAPα-cleavable pro-peptides. According to some embodiments, the pro-peptides comprise a membrane interacting domain, and a masking domain that, when linked to the membrane interacting domain, is effective to inhibit interaction of the membrane interacting domain with a phospholipid bilayer. Such pro-peptides further comprise a FAPα cleavage site disposed between the membrane interacting domain and the masking domain.

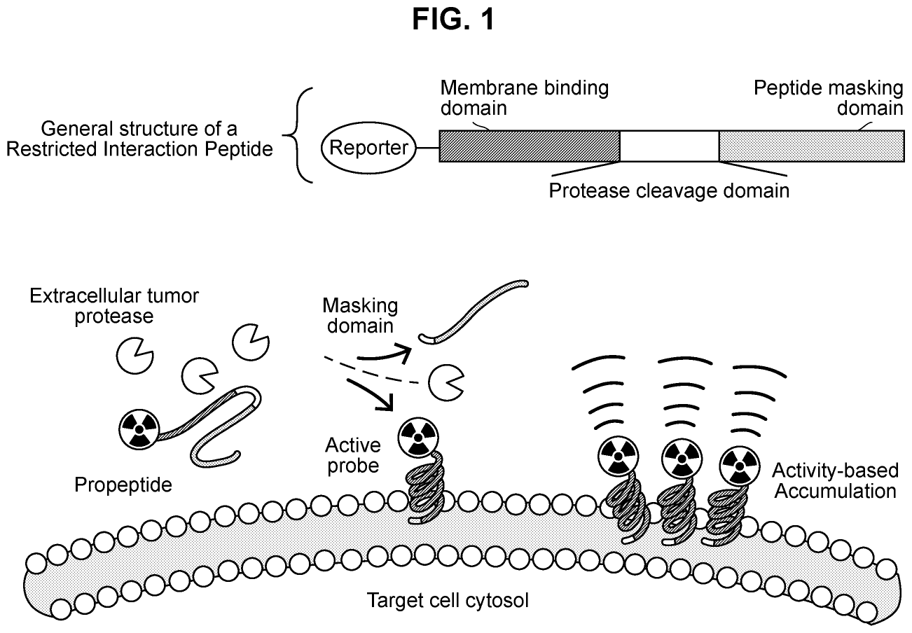

A schematic illustration of a pro-peptide (sometimes referred to herein as a “restricted interaction peptide” or “RIP”) according to embodiments of the present disclosure is provided in FIG. 1. RIPs may be low MW (˜4 kDa) peptides consisting of three domains from N- to C-terminus: (i) a membrane interacting domain (in this example, a membrane binding antimicrobial peptide (AMP)) coupled to a payload (e.g., a chelator bound to a radioisotope); (ii) an endoprotease cleavage site, e.g., spanning P4-P4′; and (iii) a peptide “masking domain” that prevents the AMP from adopting a helical conformation required for membrane binding. Bottom: In this example, upon cleavage of the pro-peptide by the target endoprotease in vivo, the radiolabeled AMP is liberated, spontaneously adopts a helical conformation and immediately attaches to a nearby phospholipid membrane.

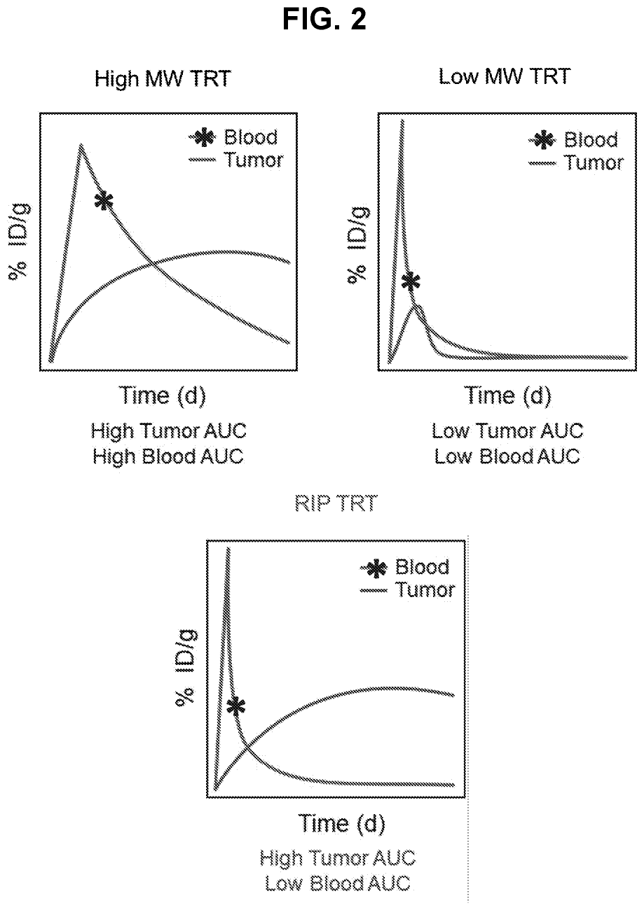

The pro-peptides of the present disclosure find use in a variety of contexts. For example, when the membrane interacting domain is stably associated with (e.g., conjugated to) one or more therapeutic agents (e.g., a radioisotope), the pro-peptides find use in treating conditions associated with FAPα expression in subjects in need thereof. Non-limiting examples of such conditions include cancer. FIG. 2 shows a schema illustrating the advantage of RIP-based targeted radiotherapy (TRT). At left is shown the typical blood and tumor activity curve for large molecular weight targeted radiotherapies (e.g., immunoglobulins). High MW targeted radiotherapies have high tumor uptake but dose limiting toxicity due to slow clearance from blood. Low MW targeted radiotherapies (e.g., small molecule radioligand therapies) benefit from improved safety due to rapid serum clearance but rapid clearance by nature limits tumor exposure. RIPs achieve both desirable safety due to rapid clearance and high tumoral AUC due to the renewable, catalytic amplification of isotope accumulation in tumors as well as the persistent binding of membrane interacting peptides to cell membranes.

Also by way of example, when the membrane interacting domain is detectably labeled (e.g., with a radioisotope), the pro-peptides find use in assessing for FAPα activity in a subject, e.g., by in vivo imaging for cells labeled with the detectably labeled membrane interacting domains. As demonstrated in the Experimental section herein, the pro-peptides of the present disclosure surprisingly exhibit superior biodistribution as compared to an active site directed FAPα radioligand currently in clinical trials. Details regarding the pro-peptides of the present disclosure will now be described.

The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof. The series of amino acid residues can include genetically coded and non-genetically coded amino acids, chemically or biochemically modified or derivatized amino acids, and peptides having modified peptide backbones.

The term “pro-peptide” as used herein refers to a peptide whose activity is restricted because the individual portions of the peptide are linked together, therefore limiting or restricting the activity that the individual portions may have when not linked to one another. The activity of the individual portions of a pro-peptide is unleashed upon cleavage or disruption of one or more bonds that hold the individual portions together.

According to some embodiments, a pro-peptide of the present disclosure comprises from to 50 amino acids. For example, in some embodiments, a pro-peptide of the present disclosure comprises at least 10 amino acids, but 50 or fewer, 45 or fewer, 40 or fewer, 35 or fewer, or 30 or fewer amino acids.

In certain embodiments, a pro-peptide of the present disclosure comprises from N-terminal to C-terminal: the membrane interacting domain, the FAPα cleavage site, and the masking domain. As used herein in the context of the structure of a polypeptide, “N-terminus” and “C-terminus” refer to the extreme amino and carboxyl ends of the polypeptide, respectively, while “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

### Membrane-Interacting Domains

In certain embodiments, the membrane interacting domain (sometimes referred to herein as a “membrane binding domain”) comprises a plurality of nonpolar hydrophobic amino acid residues, and, when unconstrained by the masking domain, comprises an alpha-helical structure capable of interaction with phospholipid bilayers such as a cell membrane. Such secondary structure may appear before, during or after insertion of the membrane interacting domain into the phospholipid bilayer. The composition of membrane interacting domains as described herein is not strictly limited to nonpolar hydrophobic amino acid residues, as such peptides may include different types of amino acid residues, for example, polar uncharged, polar basic, or polar acidic amino acid residues as well.

As such, in some instances, the membrane interacting domain, when separated from the masking domain, comprises an alpha-helical structure capable of inserting into a phospholipid bilayer. An alpha helix is a common motif in the secondary structure of proteins, and generally comprises a right-handed coiled or spiral conformation that is stabilized by hydrogen bonds in which the N—H group of a first amino acid residue forms a hydrogen bond with the C═O group of an amino acid residue located four residues away in the polypeptide chain. A typical alpha helix comprises approximately 3.6 amino acid residues per turn of the helix, and is a tightly-packed structure. The side chains of the amino acid residues that make up an alpha helix face the outside of the helix. Different amino acid sequences have different propensities for forming alpha helices due, in part, to the differing chemical properties of the amino acid side chains.

According to some embodiments, the membrane interacting domain comprises about 5 to about 30 amino acid residues. For example, in some embodiments, the membrane interacting domain comprises at least 5 amino acids, but 30 or fewer, 25 or fewer, 20 or fewer, or 15 or fewer amino acid residues.

In certain embodiments, the membrane interacting domain comprises an anti-microbial peptide (AMP) or portion thereof. The AMP or portion thereof may be incorporated into the pro-peptides of the present disclosure in their naturally-occurring form, or may be modified to alter their chemical properties and adapt such for a desired use. For example, the membrane-interaction potential of antimicrobial peptides may be strengthened or weakened by, e.g., adding, eliminating or substituting certain amino acid residues in the protein sequence. Such additions, eliminations, or substitutions can be made, e.g., to introduce charged amino acid residues, to eliminate charged amino acid residues, to introduce hydrophobic amino acid residues, to eliminate hydrophobic amino acid residues, etc.

According to some embodiments, an antimicrobial peptide sequence may be altered by chemically modifying the peptide with disulfide bonds or other chemical modifications (e.g., amidation). Many antimicrobial peptides are naturally produced with such modifications to improve the potency of their interactions with phospholipid membranes and resistance to proteolysis.

In some embodiments, the membrane interacting domain comprises a protein from the Temporin family. Proteins in the Temporin family generally range from about 10 up to about 14 amino acids in length. The consensus sequence for the Temporin family of proteins showing the most abundant amino acid found at each position is: FLP(I/L)IASLL(S/G)KLL (SEQ ID NO:1). The consensus sequence for the Temporin family of proteins showing the general amino acid type found at each position is: X^{a}X^{b}X^{c}X^{d}X^{e}X^{f}Y^{a}X^{g}X^{h}Y^{b}Y*XⁱX^{j}, where X^{a}, X^{b}, X^{c}, X^{d}, X^{e}, X^{f}, X^{g}, X^{h}, Xⁱ, and X^{j} are hydrophobic amino acid residues, Y^{a} and Y^{b} are hydrophilic amino acid residues, and Y is a charged amino acid residue. The table below shows the amino acid sequences of several Temporin and Temporin-like peptides that are useful in the pro-peptides and methods of the present disclosure.

As described above, antimicrobial peptide sequences may be altered by eliminating or substituting one or more of the amino acid residues. For example, in some embodiments, the membrane interacting domain comprises Temporin-L, whose amino acid sequence is FVQWFSKFLGRIL (SEQ ID NO:2). In other embodiments, the membrane interacting domain comprises a derivative of Temporin-L having the amino acid sequence FVQWFSKFLGKLL (SEQ ID NO:3), wherein amino acid residues R and I at positions 11 and 12 of the Temporin-L sequence have been replaced with amino acid residues K and L, respectively.

TABLE 1
Amino acid sequences of Temporin and Temporin-like peptides
thirteen amino acids in length.

| Peptide | | | | | | | | | | | | | | | | ID |
|---|---|---|---|---|---|---|---|---|---|---|---|---|---|---|---|---|
| Name | Amino Acid Sequence | | | | | | | | | | | | | | | NO |
| Temporin-A | — | F | L | P | L | I | G | R | V | L | S | G | I | L | — | 4 |
| Temporin-B | — | L | L | P | I | V | G | N | L | L | K | S | L | L | — | 5 |
| Temporin-C | — | L | L | P | I | L | G | N | L | L | N | G | L | L | — | 6 |
| Temporin-D | — | L | L | P | I | V | G | N | L | L | N | S | L | L | — | 7 |
| Temporin-E | — | V | L | P | I | I | G | N | L | L | N | S | L | L | — | 8 |
| Temporin-F | — | F | L | P | L | I | G | K | V | L | S | G | I | L | — | 9 |
| Temporin-G | — | F | F | P | V | I | G | R | I | L | N | G | I | L | — | 10 |
| Temporin-H | — | L | S | P | — | — | — | N | L | L | K | S | L | L | — | 11 |
| Temporin-K | — | L | L | P | — | — | — | N | L | L | K | S | L | L | — | 12 |
| Temporin-L | — | F | V | Q | W | F | S | K | F | L | G | R | I | L | — | 2 |
| Temporin-1Ca | — | F | L | P | F | L | A | K | I | L | T | G | V | L | — | 13 |
| Temporin-1Cb | — | F | L | P | L | F | A | S | L | I | G | K | L | L | — | 14 |
| Temporin-1Cc | — | F | L | P | F | L | A | S | L | L | T | K | V | L | — | 15 |
| Temporin-1Cd | — | F | L | P | F | L | A | S | L | L | S | K | V | L | — | 16 |
| Temporin-1Ce | — | F | L | P | F | L | A | T | L | L | S | K | V | L | — | 17 |
| Temporin-1Ga | S | I | L | P | T | I | V | S | F | L | S | K | V | F | — | 18 |
| Temporin-1Gb | S | I | L | P | T | I | V | S | F | L | S | K | F | L | — | 19 |
| Temporin-1Gc | S | I | L | P | T | I | V | S | F | L | T | K | FL | | — | 20 |
| Temporin-1Gd | F | I | L | P | L | I | A | S | F | L | S | K | F | L | — | 21 |
| Temporin-1La | — | V | L | P | L | I | S | M | A | L | G | K | L | L | — | 22 |
| Temporin-1Lb | N | F | L | G | T | L | I | N | L | A | K | K | I | M | — | 23 |
| Temporin-1Lc | — | F | L | P | I | L | I | N | L | I | H | K | G | L | L | 24 |
| Temporin-1P | — | F | L | P | I | V | G | K | L | L | S | G | L | L | — | 25 |
| Ranatuerin-5 | — | F | L | P | I | — | A | S | L | L | G | K | Y | L | — | 26 |
| Ranatuerin-6 | — | F | I | S | A | I | A | S | M | L | G | K | F | L | — | 27 |
| Ranatuerin-7 | — | F | L | S | A | I | A | S | M | L | G | K | F | L | — | 28 |
| Ranatuerin-8 | — | F | I | S | A | I | A | S | F | L | G | K | F | L | — | 29 |
| Ranatuerin-9 | F | L | F | P | L | I | T | S | F | L | S | K | V | L | — | 30 |
| Peptide A1 | — | F | I | P | A | I | A | G | | L | S | Q | L | F | — | 31 |
| Peptide B9 | — | F | L | P | L | I | A | G | L | L | G | K | L | F | — | 32 |

Longer and shorter members of the family have also been described but are not included in this table.

In some embodiments of the present disclosure, a membrane interacting domain comprises a Temporin or a Temporin-like peptide listed in Table 1, or a conservative amino acid substitution thereof. In some embodiments of the present disclosure, a membrane interacting domain comprises the sequence of Temporin-L (FVQWFSKFLGRIL; SEQ ID NO:2), or a conservative amino acid variant thereof.

In some embodiments of the present disclosure, a membrane interacting domain comprises Protonectin, having the amino acid sequence ILGTILGLLKGL (SEQ ID NO:33), or a conservative amino acid variant thereof.

In some embodiments, a membrane interacting domain may comprise a Japonicin or a Japonicin-like peptide. In some embodiments of the present disclosure, a membrane-interacting peptide comprises the sequence of Japonicin-1 (FFPIGVFCKIFKTC; SEQ ID NO:34), or a conservative amino acid variant thereof. Japonicins are naturally obtainable from the skin of the Japanese brown frog Rana japonica and range in length from about 14 up to about 21 amino acid residues.

### Masking Domains

As summarized above, the pro-peptides of the present disclosure comprise a masking domain that, when linked to the membrane interacting domain, is effective to inhibit interaction of the membrane interacting domain with a phospholipid bilayer.

In certain embodiments, the masking domain comprises from 5 to 30 amino acids. For example, in some embodiments, the masking domain comprises at least 5 amino acids, but 30 or fewer, 25 or fewer, 20 or fewer, 15 or fewer, 14 or fewer, 13 or fewer, 12 or fewer, 11 or fewer, or 10 or fewer amino acid residues.

According to some embodiments, the masking domain comprises a peptide derived from protease-activated receptor-1 (PAR-1). “Derived from” in the context of an amino acid sequence or polynucleotide sequence is meant to indicate that the polypeptide or nucleic acid has a sequence that is based on that of a reference polypeptide or nucleic acid, and is not meant to be limiting as to the source or method in which the protein or nucleic acid is made.

In certain embodiments, when the masking domain comprises a peptide derived from PAR-1, the masking domain comprises the amino acid sequence QDPNDQYEPF (SEQ ID NO:35). In other embodiments, when the masking domain comprises a peptide derived from PAR-1, the masking domain comprises the amino acid sequence RNPNDKYEPF (SEQ ID NO:36).

### FAPα Cleavage Sites

As summarized above, the pro-peptides of the present disclosure comprise a FAPα cleavage site disposed between the membrane interacting domain and the masking domain. According to some embodiments, the FAPα cleavage site comprises the amino acid sequence YHGPLAHX (SEQ ID NO:37), HIGPTAAY (SEQ ID NO:38), XXIPTNIR (SEQ ID NO:39), HQGPFWML (SEQ ID NO:40), XXGPKLTY (SEQ ID NO:41), HYGPTVNK (SEQ ID NO:42), XXWPMGMY (SEQ ID NO:43), XXFPNMWS (SEQ ID NO:44), XXGSQVFS (SEQ ID NO:45), XXMPEEVA (SEQ ID NO:46), or XXHPTKSF (SEQ ID NO:47), wherein X is any amino acid.

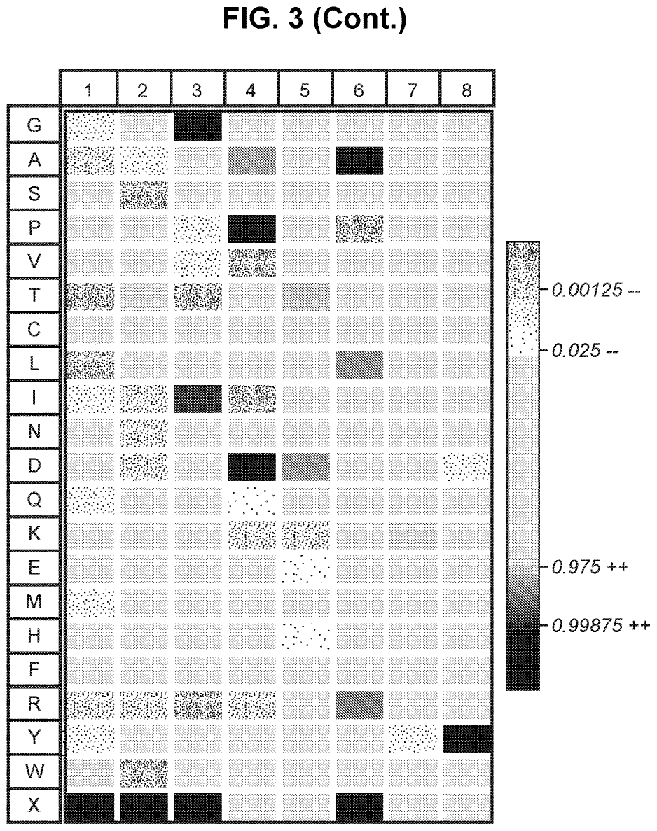

In certain embodiments, the FAPα cleavage site comprises the FAPα specific motif GP in the P2-P1 positions. For example, FAPα cleavage sites that may be incorporated into a pro-peptide of the present disclosure include, but are not limited to, a FAPα cleavage site comprising the sequence HQGPFWML (SEQ ID NO:40), XXGPKLTY (SEQ ID NO:41), or HYGPTVNK (SEQ ID NO:42). According to some embodiments, the FAPα cleavage site comprises the sequence HQGPFWML (SEQ ID NO:40). As demonstrated in the Experimental section and FIG. 4 herein, the kcat/Km for a FAPα cleavage site comprises the sequence HQGPFWML (SEQ ID NO:40) was found to be ˜80,000 M⁻¹ sec⁻¹, the highest reported turnover number to date for FAPα substrates.

### Therapeutic Agents

As noted above, when the membrane interacting domain of a pro-peptide of the present disclosure is stably associated with (e.g., conjugated to) one or more therapeutic agents, the pro-peptides find use in treating conditions associated with FAPα expression (e.g., cancers associated with FAPα expression) in subjects in need thereof.

Accordingly, in certain embodiments, provided are pro-peptides comprising a membrane interacting domain stably associated with (e.g., conjugated to) one or more therapeutic agents. As used herein, a “therapeutic agent” is a physiologically or pharmacologically active substance that can produce a desired biological effect in a targeted site in an animal, such as a mammal or in a human. The therapeutic agent may be any inorganic compound, organic compound, radioisotope, or the like. A therapeutic agent may decrease, suppress, attenuate, diminish, arrest, or stabilize the development or progression of disease, disorder, or cell growth in an animal such as a mammal or human. Examples include, without limitation, peptides, proteins, nucleic acids (including siRNA, miRNA and DNA), polymers, small molecules, and radioisotopes. When the function of the cell/tissue is pathological, a therapeutic agent that reduces the function of the cell/tissue may be employed. In certain embodiments, a membrane interacting domain is stably associated with an agent that reduces the function of a target cell/tissue by inhibiting cell proliferation and/or killing the cell/tissue. Such agents may vary and include radioisotopes, cytostatic agents and cytotoxic agents, e.g., an agent capable of killing a target cell tissue with or without being internalized into a target cell.

In certain embodiments, “stably associating” means a physical association between two entities in which the mean half-life of association is one day or more in PBS at 4° C. In some embodiments, the physical association between the two entities has a mean half-life of one day or more, one week or more, one month or more, including six months or more, e.g., 1 year or more, in PBS at 4° C. According to some embodiments, the stable association arises from a covalent bond between the two entities, a non-covalent bond between the two entities (e.g., an ionic or metallic bond), or other forms of chemical attraction, such as hydrogen bonding, Van der Waals forces, and the like. In certain embodiments, the membrane interacting domain is conjugated to one or more therapeutic agents.

According to some embodiments, the one or more therapeutic agents comprise a cytotoxic agent, a toxin, a radiation-sensitizing agent, a radioisotope, or any combination thereof.

In certain embodiments, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to some embodiments, the agent is a toxin, such as a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the agent is a radiation-sensitizing agent. As used herein, a “radiation-sensitizing agent” is an agent that enhances the ability of radiation to kill cells (e.g., tumor cells). Non-limiting examples of radiation-sensitizing agents that may be conjugated to a membrane interacting domain include cisplatin, 5-fluorouracil (5-FU), AZD7762, selumetinib, and the like.

In certain embodiments, the agent is a radioisotope, e.g., useful for therapy and/or detection (e.g., imaging). Non-limiting examples of radioisotopes that may be conjugated to a membrane interacting domain include but are not limited to ²²⁵Ac, ¹¹¹Ag, ¹¹⁴Ag, ⁷¹As, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ¹³C, ⁵⁵Co, ⁶²Cu, ⁶⁴Cu, ⁶⁷Cu, ¹⁶⁵Dy, ¹⁶⁶Dy, ¹⁶⁹Er, ¹⁸F, ¹⁹F, ⁵²Fe, ⁵⁹Fe, ⁶⁶Ga, ⁶⁷Ga, ⁶⁸Ga, ⁷²Ga, ¹⁵⁴⁻¹⁵⁸Gd, ¹⁵⁷Gd, ¹⁵⁹Gd, ¹⁶⁶Ho, ¹²⁰I, ¹²¹I, ¹²³I, ¹²⁴I, ¹²⁵I, ¹³¹I, ¹⁰⁹In, ¹¹¹In, ^{133m}In, ^{81m}Kr, ¹⁷⁷Lu, ⁵¹Mn, ⁵²Mn, ⁹⁹Mo, ¹³N, ¹⁵N, ¹⁵O, ¹⁷O, ³²P, ³³P, ²¹¹Pb, ²¹²Pb, ¹⁰⁹Pd, ¹⁴⁹Pm, ¹⁵¹Pm, ¹⁴²Pr, ¹⁴³Pr, ¹⁹¹PT, ^{193m}PT, ^{195m}Pt, ²²³Ra, ¹⁴²Rb, ¹⁸⁶Re, ¹⁸⁸Re, ¹⁸⁹Re, ¹⁰⁵Rh, ⁴⁷Sc, ⁷⁵Se, ¹⁵³Sm, ^{117m}Sn, ¹²¹Sn, ⁸³Sr, ⁸⁹Sr, ¹⁶¹Tb, ⁹⁴Tc, ⁹⁹Tc, ^{99m}Tc, ²²⁷Th, ²⁰¹Tl, ¹⁷²Tm, ¹²⁷Te, ⁹⁰Y, ¹⁶⁹Yb, ¹⁷⁵Yb, ¹³³X, and ⁸⁹Zr. According to some embodiments, the membrane interacting domain is conjugated to ⁶⁴Cu (Copper-64), ¹⁷⁷Lu (Lutetium-177), or ²²⁵Ac (Actinium-225).

In certain embodiments, a radioisotope is conjugated to the membrane interacting domain via a chelator. Non-limiting examples of a chelators that may be employed include 1,4,7,10-tetraazacyclododecane-1,4,7,10-tetraacetic acid (DOTA), diethylene triamine pentaacetic acid (DTPA), MACROPA, and the like.

### Methods of Producing Pro-Peptides

A pro-peptide of the present disclosure may be produced by any suitable method, including recombinant and non-recombinant methods (e.g., chemical synthesis). Where a polypeptide is chemically synthesized, the synthesis may proceed via liquid-phase or solid-phase. Solid-phase synthesis (SPPS) allows the incorporation of unnatural amino acids, peptide/protein backbone modification. Various forms of SPPS, such as Fmoc and Boc, are available for synthesizing pro-peptides of the present disclosure. Details of the chemical synthesis are known in the art (e.g., Ganesan A. 2006 Mini Rev. Med Chem. 6:3-10 and Camarero J A et al. 2005 Protein Pept Lett. 12:723-8). Briefly, small insoluble, porous beads are treated with functional units on which peptide chains are built. After repeated cycling of coupling/deprotection, the free N-terminal amine of a solid-phase attached peptide or amino acid is coupled to a single N-protected amino acid unit. This unit is then deprotected, revealing a new N-terminal amine to which a further amino acid may be attached. The peptide remains immobilized on the solid-phase and undergoes a filtration process before being cleaved off.

In one non-limiting example, a pro-peptide of the present disclosure may be synthesized by Fmoc solid phase synthesis on a Biotage Syroll peptide synthesizer at ambient temperature. The synthesis scale may be at 12.5 μM using preloaded lysine(2-dinitrophenyl) Wang resin where the DNP quencher may be linked to the epsilon nitrogen of the lysine. Coupling reactions may be carried out with 4.9 equivalents of HCTU (O-(1H-6-chlorobenzotriazole-1-yl)-1,1,3,3-tetramethyluronium hexafluoro-phosphate), 5 equivalents of Fmoc-amino acid-OH, and 20 equivalents of N-methylmorpholine (NMM) in 500 μL N,N-dimethylformamide (DMF) for 8 minutes while shaking. Each amino acid position may be double coupled and subsequent Fmoc deprotection may be carried out with 500 μL of 40% 4-methylpiperadine in DMF for 10-minutes followed by 6 washes with 500 μL DMF for 3-minutes. Pro-peptides may be cleaved from Wang's resin with 500 μL of solution composed of 95% trifluoroacetic acid, 2.5% water, and 2.5% triisopropylsilane for 1-hour while shaking. Crude pro-peptide product may then be precipitated in 30 mL cold 1:1 diethyl ether: hexanes and then solubilized in a 1:1:1 mixture of DMSO:water:acetonitrile. Solubilized crude may be purified by high-performance liquid chromatography (HPLC) using an Agilent Pursuit 5 C18 column (5 mm bead size, 150×21.2 mm) on an Agilent PrepStar 218 series preparative HPLC. Mobile phase A and B may be water+0.1% TFA and acetonitrile+0.1% TFA, respectively. Solvent may be removed under reduced atmosphere and purified peptide product may be solubilized into a DMSO stock at a desired final concentration, e.g., 10 mM. Purity may be confirmed by liquid chromatography-mass spectrometry and the stock stored at −20° C.

Where the pro-peptide is produced using recombinant techniques, the pro-peptide may be produced as an intracellular protein or as a secreted protein, using any suitable construct and any suitable host cell, which can be a prokaryotic or eukaryotic cell, such as a bacterial (e.g., E. coli) or a yeast host cell, respectively.

Other examples of eukaryotic cells that may be used as host cells include insect cells, mammalian cells, and/or plant cells. Where mammalian host cells are used, the cells may include one or more of the following: human cells (e.g., HeLa, 293, H9 and Jurkat cells); mouse cells (e.g., X3, NIH3T3, pancreatic ductal adenocarcinoma 2.1, L cells, and C127 cells); primate cells (e.g., Cos 1, Cos 7 and CV1) and hamster cells (e.g., Chinese hamster ovary (CHO) cells).

A wide range of host-vector systems suitable for the expression of the subject pro-peptide may be employed according to standard procedures known in the art. See, e.g., Sambrook et al. 1989 Current Protocols in Molecular Biology Cold Spring Harbor Press, New York and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are available commercially.

Vectors can provide for extrachromosomal maintenance in a host cell or can provide for integration into the host cell genome. The expression vector provides transcriptional and translational regulatory sequences, and may provide for inducible or constitutive expression, where the coding region is operably linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like). Expression constructs generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture, by immunoaffinity purification, which generally involves contacting the sample with an anti-protein antibody, washing to remove non-specifically bound material, and eluting the specifically bound protein. The isolated protein can be further purified by dialysis and other methods normally employed in protein purification methods. In one embodiment, the protein may be isolated using metal chelate chromatography methods. Protein of the present disclosure may contain modifications to facilitate isolation.

The subject pro-peptides may be prepared in substantially pure or isolated form (e.g., free from other polypeptides). The pro-peptide can be present in a composition that is enriched for the polypeptide relative to other components that may be present (e.g., other polypeptides or other host cell components). Purified pro-peptide may be provided such that the pro-peptide is present in a composition that is substantially free of other expressed proteins, e.g., less than 98%, less than 95%, less than 90%, less than 80%, less than 60%, or less than 50%, of the composition is made up of other expressed proteins.

DOTA-FRIP may be synthesized first using solid phase peptide synthesis conditions as outlined above. Resin-bound peptide with N-terminal hexanoic acid may then be triple coupled with two equivalents of dota-NHS, five equivalents of HCTU, and twenty equivalents of N,N-Diisopropylethylamine (DIPEA) for 12 hours to synthesize DOTA-FRIP.

### Compositions

Aspects of the present disclosure also include compositions. According to some embodiments, a subject composition comprises any of the pro-peptides of the present disclosure, including but not limited to any of the pro-peptides described in the Pro-Peptides section above and in the Experimental section below.

In certain aspects, a composition of the present disclosure includes the pro-peptide present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure comprises a pro-peptide of the present disclosure, and a pharmaceutically acceptable carrier.

The pro-peptides can be incorporated into a variety of formulations for therapeutic, diagnostic, or theranostic administration. More particularly, the pro-peptides can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the pro-peptides for administration to an individual (e.g., suitable for human administration) are generally sterile and may further be free of detectable pyrogens or other contaminants contraindicated for administration to a patient according to a selected route of administration.

In pharmaceutical dosage forms, the pro-peptides can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the pro-peptides can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pro-peptides can be formulated for parenteral (e.g., intravenous, intra-arterial, intraosseous, intramuscular, intracerebral, intracerebroventricular, intrathecal, subcutaneous, intra-tumoral, etc.) administration. In certain aspects, the pro-peptides are formulated for injection by dissolving, suspending or emulsifying the pro-peptides in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the pro-peptides may be prepared by mixing the pro-peptides having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the pro-peptides may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the pro-peptide against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the pro-peptide, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure also include kits. According to some embodiments, a subject kit comprises any of the pro-peptides of the present disclosure, including but not limited to any of the pro-peptides described in the Pro-Peptides section above and in the Experimental section below.

In certain embodiments, the kits find use in practicing the methods of the present disclosure, e.g., methods of treating a condition associated with FAPα expression in a subject in need thereof, methods of assessing for FAPα activity in a subject, and/or the like.

Accordingly, in certain embodiments, a kit of the present disclosure may comprises any of the pro-peptides or pharmaceutical compositions of the present disclosure, and instructions for administering the pro-peptide or pharmaceutical composition to a subject in need thereof.

The kits of the present disclosure may include a quantity of the pro-peptide or pharmaceutical composition, present in unit dosages, e.g., ampoules, or a multi-dosage format. As such, in certain embodiments, the kits may include one or more (e.g., two or more) unit dosages (e.g., ampoules) of a composition that includes a pro-peptide of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular pro-peptide employed, the effect to be achieved, and the pharmacodynamics associated with the pro-peptide, in the subject. In yet other embodiments, the kits may include a single multi dosage amount of the composition.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Use

Aspects of the present disclosure include methods of using the pro-peptides of the present disclosure. The methods are useful in a variety of contexts, including in vitro and/or in vivo research and/or clinical applications.

In certain aspects, provided are methods of treating a condition associated with FAPα expression in a subject in need thereof, the method comprising administering an effective amount of a pro-peptide of the present disclosure to the subject.

In some embodiments, the condition associated with FAPα expression is cancer. The terms “cancer” and “cancerous” refer to or describe the physiological condition in mammals that is typically characterized by unregulated cell growth/proliferation. The subject methods may be employed for the treatment of a large variety of cancers. Normal tissues have low and generally undetectable levels of FAPα expression. However, FAPα is overexpressed in many tumor tissues, including breast, colorectal, pancreatic, lung, brain, intrahepatic bile duct, and ovarian cancers. In addition, high levels of FAPα expression can be detected in some tumors that are derived from non-epithelial tissues, such as melanoma and myeloma. In these tumors, FAPα overexpression is often observed in the interstitium, which has led to FAPα being considered a universal marker for CAFs, although FAPα expression can also be detected in gastric carcinoma, pancreatic carcinoma and melanoma cells. Details regarding FAPα expression in human malignancies can be found, e.g., in Busek et al. (2018) Frontiers In Bioscience, Landmark 23:1933-1968.

Accordingly, in some embodiments, when the condition associated with FAPα expression is cancer, the cancer may be characterized by FAPα expression on cancer cells, cancer associated fibroblasts, or both. In certain embodiments, the cancer comprises a solid tumor. Examples of solid tumors treatable by the methods of the present disclosure include carcinomas, lymphomas, blastomas, and sarcomas. Non-limiting examples of cancers treatable by the methods of the present disclosure include basal cell carcinoma, squamous cell carcinoma of the skin, oral squamous cell carcinoma, melanoma, esophageal cancer, gastric cancer, colorectal cancer, pancreatic adenocarcinoma, hepatocellular carcinoma, non-small cell lung cancer, mesothelioma, breast cancer, renal cancer, prostate cancer, cervical cancer, ovarian cancer, glioma, parathyroid cancer, a sarcoma, and a myeloma.

In certain embodiments, when the condition associated with FAPα expression is cancer, the cancer may comprise a hematological malignancy. According to some embodiments, the hematological malignancy is a leukemia, a lymphoma, or multiple myeloma.

Conditions associated with FAPα expression other than cancers are also treatable by the methods of the present disclosure. For example, the condition associated with FAPα expression may be a fibrotic disease. In some embodiments, the fibrotic disease comprises liver fibrosis. According to certain embodiments, the fibrotic disease is interstitial lung disease.

The pro-peptides of the present disclosure may be administered in a composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer, fibrotic disease, or other condition associated with FAPα expression, as compared to a control. With respect to cancer, in some embodiments, the therapeutically effective amount is sufficient to slow the growth of a tumor, reduce the size of a tumor, and/or the like. An effective amount can be administered in one or more administrations.

As described above, aspects of the present disclosure include methods for treating a condition associated with FAPα expression in a subject in need thereof. By “treatment” or “treating” is meant at least an amelioration of one or more symptoms associated with the condition associated with FAPα expression (e.g., cancer, fibrotic disease, or the like) where amelioration is used in a broad sense to refer to at least a reduction in the magnitude of a parameter, e.g. symptom, associated with the condition being treated. As such, treatment also includes situations where the condition, or at least one or more symptoms associated therewith, are completely inhibited, e.g., prevented from happening, or stopped, e.g., terminated, such that the subject no longer suffers from the condition, or at least the symptoms that characterize the condition.

A pro-peptide of the present disclosure may be administered to the subject alone or in combination with a second agent. Second agents of interest include, but are not limited to, agents approved by the United States Food and Drug Administration and/or the European Medicines Agency (EMA) for use in treating conditions associated with FAPα expression, such as cancer, fibrotic disease, or the like.

Also provided by the present disclosure are methods of detectably labeling a phospholipid bilayer of a cell in the presence of FAPα activity. Such methods comprise contacting a pro-peptide of the present disclosure with FAPα contributing to the FAPα activity. The membrane interacting domain is detectably labeled, and the FAPα cleavage site of the pro-peptide is cleaved by the FAPα to release a cleavage product comprising the detectably labeled membrane interacting domain, such that the detectably labeled membrane interacting domain interacts with the phospholipid bilayer of the cell and detectably labels the phospholipid bilayer of the cell in the presence of FAPα activity.

Aspects of the present disclosure further include methods of assessing for FAPα activity in a subject. Such methods comprise administering a pro-peptide of the present disclosure to the subject, wherein the membrane interacting domain is detectably labeled, wherein at sites of FAPα activity in the subject, the FAPα cleavage site of the pro-peptide is cleaved by FAPα to release cleavage products comprising the detectably labeled membrane interacting domain, and wherein the cleavage products interact with the phospholipid bilayers of cells at the sites of FAPα activity in the subject. Such methods further comprise assessing for the presence or absence of cells labeled with the cleavage products, wherein the presence of cells labeled with the cleavage products indicates FAPα activity in a subject.

In certain embodiments, the membrane interacting domain is detectably labeled with a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 8000W Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, ¹⁷⁷Lu, ²²⁵Ac, or the like.

As will be appreciated with the benefit of the present disclosure, the pro-peptides of the present disclosure find use in theranostic applications. By way of example, when the membrane interacting domain is labeled with a radioisotope (e.g., ⁶⁴Cu (Copper-64), ¹⁷⁷Lu (Lutetium-177), or ²²⁵Ac (Actinium-225)), the pro-peptide may be employed in a method of treating a condition associated with FAPα expression (e.g., cancer, fibrotic disease, or the like) in a subject in need thereof, where the method further comprises assessing for FAPα activity in the subject utilizing the radioisotope for in vivo imaging, e.g., by PET/CT and/or PET/MR.

The following examples are offered by way of illustration and not by way of limitation.

### EXPERIMENTAL

### Example 1—Identification of Unique FAPα Substrate Sequences Using Multiplex Substrate Profiling by Mass Spectrometry (MSP-MS)

Although much is known about the substrate preferences for FAPα from P4-P1, an optimal P4-P4′ substrate sequence for FAPα has never been defined. In this example, multiplex substrate profiling by mass spectrometry (MSP-MS) was performed to identify candidate FAPα P4-P4′ substrate sequences for potential inclusion in a FAPα targeted restricted interaction peptide (or “FRIP”).

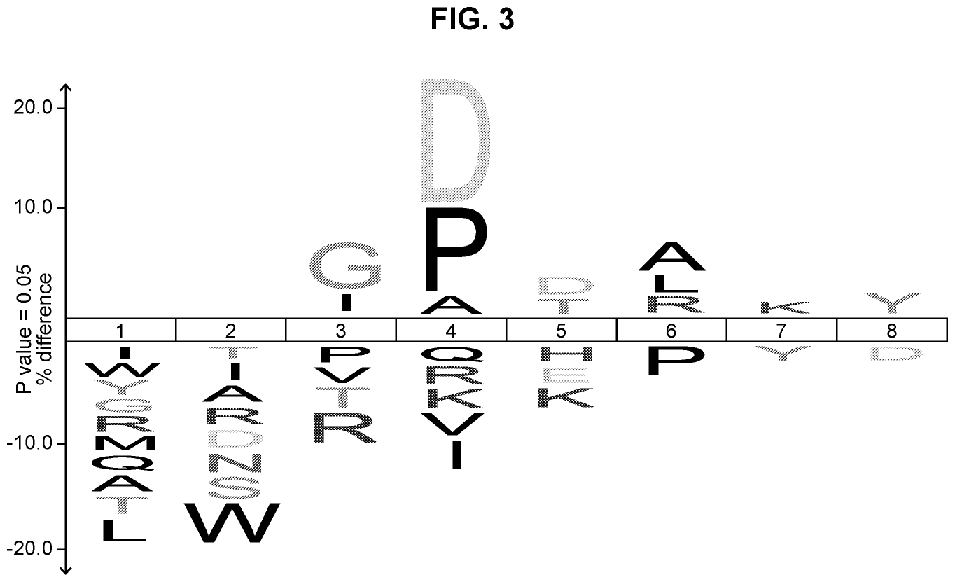

MSP-MS resulted in several potential substrates, nearly all of which bore Gly-Pro at the P2-P1 position, which was previously shown to be essential for FAPα proteolysis. Shown on the top of FIG. 3 is an Icelogo representing the cumulative amino acid preferences for recombinant human FAPα. Shown on the bottom of FIG. 3 is a heat map summarizing the amino acids that were most frequently incorporated at each site in the cleavage products. Lighter indicates an enriched amino acid. X=no preferred amino acid.

A list of the top P4-P4′ peptides cleaved by human FAP alpha as determined using the MSP-MS substrate profiling technology are shown in the following table, where X indicates an undefined amino acid site.

| P4-P4′ | SEQ | # of cleavages |
|---|---|---|
| Sequence | ID NO | on MS |
| YHGPLAHX | 37 | 98 |
| HIGPTAAY | 38 | 43 |
| XXIPTNIR | 39 | 41 |
| HQGPFWML | 40 | 30 |
| XXGPKLTY | 41 | 28 |
| HYGPTVNK | 42 | 28 |
| XXWPMGMY | 43 | 26 |
| XXFPNMWS | 44 | 23 |
| XXGSQVFS | 45 | 21 |
| XXMPEEVA | 46 | 17 |
| XXHPTKSF | 47 | 16 |
| XSGPDYQK | 48 | 14 |
| XXIARQPW | 49 | 14 |
| GMGPFHIV | 50 | 12 |

Three top hits, HIGPTAAY (SEQ ID NO:38), HQGPFWML (SEQ ID NO:40), and HYGPTVNK (SEQ ID NO:42), were selected for further analysis. An 8 mer peptide bearing FRET donor-acceptor dyes on the N and C terminus was synthesized and evaluated as a substrate for recombinant human FAPα (RND Systems) under Michaelis-Menten conditions. The kcat/Km were determined to be ˜120,000, ˜80,000 and ˜15,000 M⁻¹ sec⁻¹, respectively.

A table summarizing the kinetic properties of three of the top P4-P4′ 8 mer peptides emerging from the MSP-MS assay is provided below, where the kinetic properties were determined using FRET donor/acceptor substrate adducts and human recombinant FAP alpha.

| | SEQ | # of | | | |
|---|---|---|---|---|---|
| | ID | cleavages | k_{cat} | | k_{cat}/K_{M} |
| Probe | NO | on MSP-MS | (s⁻¹) | K_{M}(μM) | (s⁻¹M⁻¹) |
| 1. HIG | 38 | 43 | 0.2986 | 2.454 | 121679 |
| PTAAY | | | | | |
| 2. HQG | 40 | 30 | 0.1041 | 1.34 | 77687 |
| PFWML | | | | | |
| 3. HYG | 42 | 28 | 0.04031 | 2.699 | 14935 |
| PTVNK | | | | | |

Data showing kinetic properties for probe 1, probe 2 and probe 3 is provided in FIG. 4A, FIG. 4B and FIG. 4C, respectively.

### Example 2—Development of a FAPα Targeted Restricted Interaction Peptide (FRIP) and Selective Uptake in FAPα Positive Tumors In Vivo

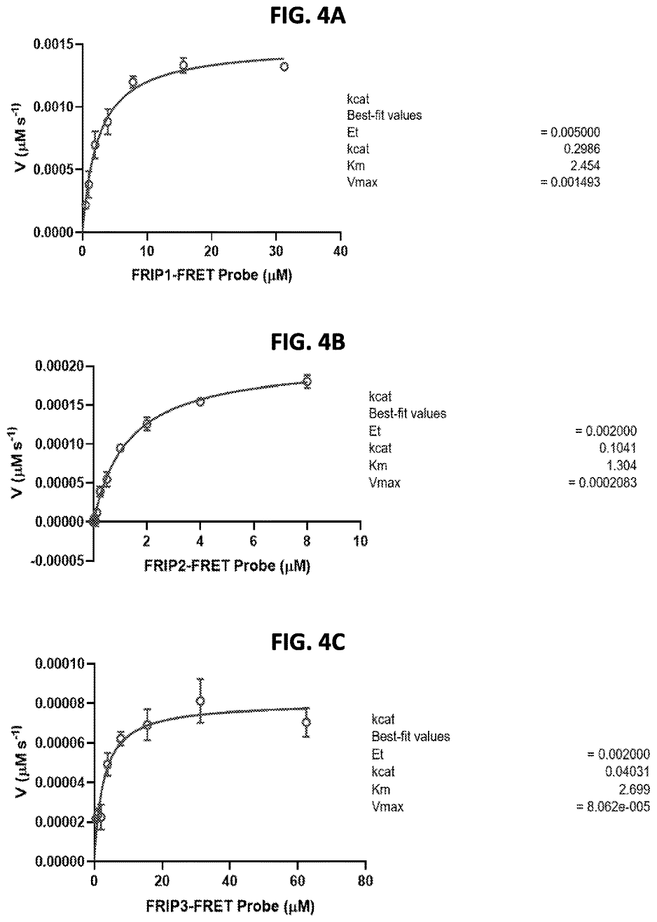

In this example, the three top cleavage sequences from the previous example were incorporated into full length RIPs bearing an N-terminal DOTA for radiolabeling (referred to herein as “FRIP1”, “FRIP2” and “FRIP3”). A schematic illustration of an example FRIP with the probe 2 sequence is provided at the top of FIG. 5. The FRIPs comprise a ⁶⁴Cu labeled membrane interacting/binding domain; (2) a FAPα cleavage site; and (3) a masking domain. In this example, the25embranee interacting domain Is an antimicrobial peptide (AMP), in particular a Temporin L peptide (FVQWFSKFLGK; SEQ ID NO:51) as the AMP. The FAPα cleavage sites are those of the top hits identified in Example 1. In this example, the masking domain is a peptide derived from protease activated receptor 1 (PAR1), the peptide having the sequence QDPNDQYEPF (SEQ ID NO:35).

DOTA-FRIPs were radiolabeled with Cu-64 using the protocol developed for DOTA-GRIP B (Zhao et al. (2021) ACS Cent Sci. 7(10):1638-49). The radiochemical yields were consistently >95% and the radiochemical purity >99%. Cleavage of each ⁶⁴Cu-FRIP by human FAPα was tested in vitro on HPLC. ⁶⁴Cu-FRIP2 cleaved most efficiently with complete conversion to a product peak within 3 hours. Lastly, the in vitro serum stability of ⁶⁴Cu-FRIP2 was tested and found to be >95% stable out to 12 hours at room temperature.

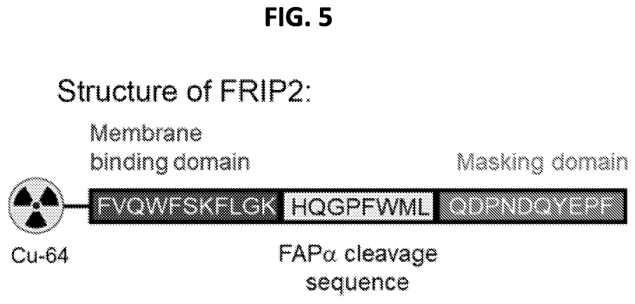

Next, compared was the uptake of ⁶⁴Cu-FRIP2 in mice with U87 xenografts to ⁶⁴Cu-FRIP1, ⁶⁴Cu-FRIP3, and ⁶⁴Cu-GRIP B. At 24 hours post injection, tumoral uptake of ⁶⁴Cu-FRIP2 was significantly higher than the other probes (FIG. 6A-6B), underscoring that efficient proteolytic cleavage is required for tumoral retention of RIPs.

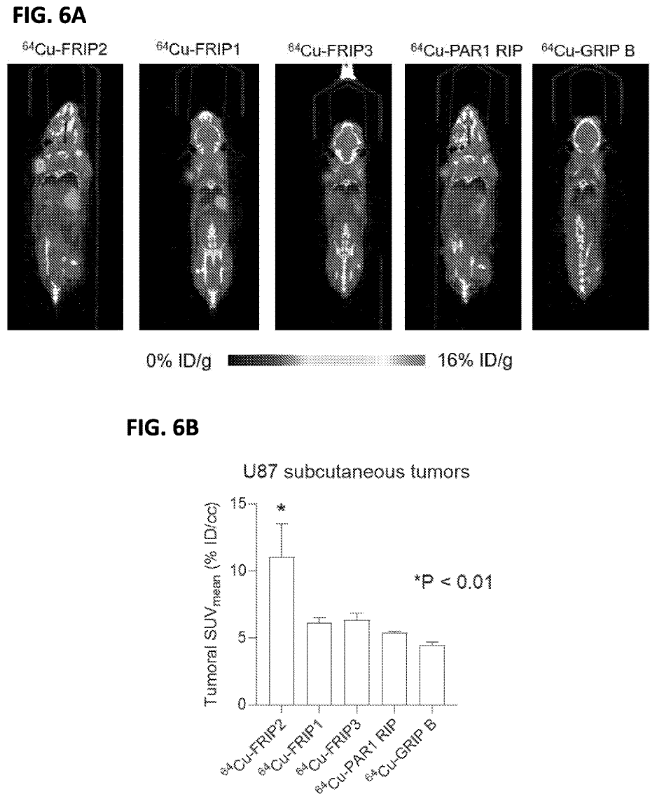

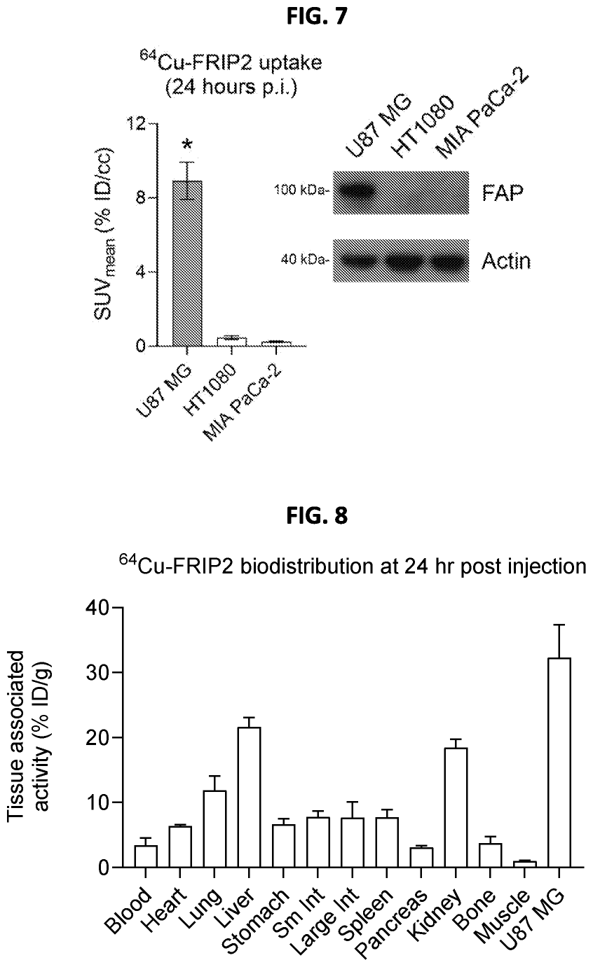

Tested next was whether ⁶⁴Cu-FRIP2 is selectively taken up by FAPα positive tumors in vivo. ⁶⁴Cu-FRIP2 (˜400 mCi/mouse, n=4 per arm) was administered to different cohorts of mice bearing subcutaneous U87 MG (FAPα positive human glioblastoma), HT1080 (FAPα negative human fibrosarcoma), or MIA Paca-2 tumors (FAPα negative human pancreatic cancer). At 24 hours post injection, the uptake of ⁶⁴Cu-FRIP2 was significantly higher in U87 tumors compared to the models lacking FAPα (FIG. 7). Biodistribution data collected at 24 hours post injection (FIG. 8) showed that U87 tumors had the highest uptake of the radiotracer compared to normal tissues. Liver and kidney also showed moderate uptake of the radiotracer. As with GRIP B, uptake in normal mouse tissues was generally low. The molecule cleared through the kidneys and had some retention in liver.

A formal dosimetry study of ⁶⁴Cu-FRIP2 in male and female C57B16 mice showed the whole body effective dose of the tracer was within range of ⁶⁴Cu-GRIP B and other commonly used tracers like ¹⁸F-FDG. The highest dose was experienced by the liver and kidneys, as we previously documented for ⁶⁴Cu-GRIP B.

### Example 3—FRIPs Exhibit Superior Biodistribution as Compared to FAPI 46

In this example, longitudinal PET studies were performed to compare the tumoral uptake of ⁶⁴Cu-FRIP2 to FAPI-46, an active site directed FAPα radioligand currently in clinical trials (Meyer et al. (2020) J Nucl Med. 61(8):1171-7). DOTA-FAPI-46 was purchased from MedChemExpress and coupled to Cu-64 using a similar protocol for ⁶⁴Cu-FRIP2. The radiochemical yield and purity were >95%.

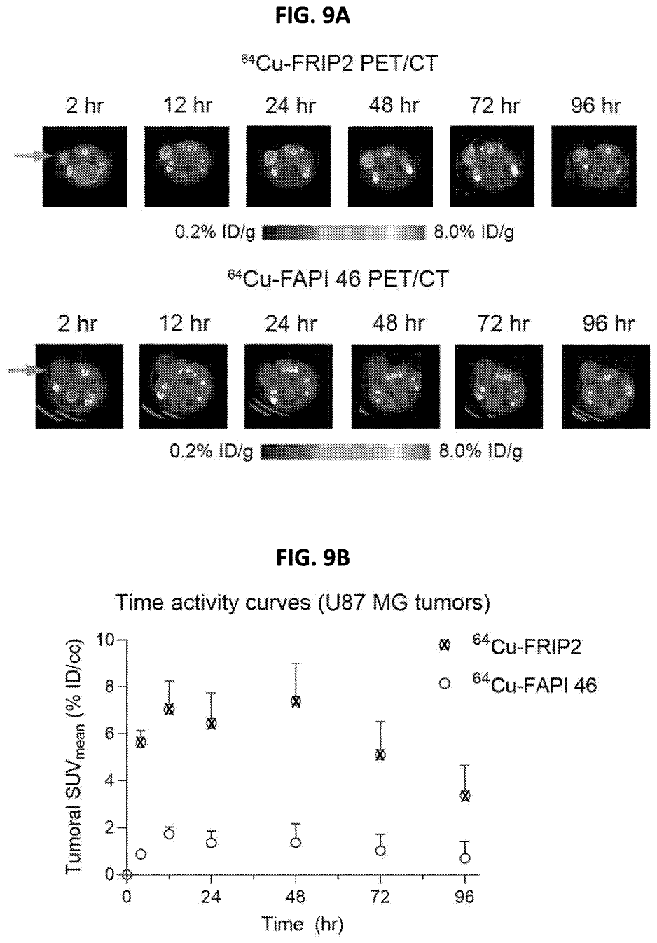

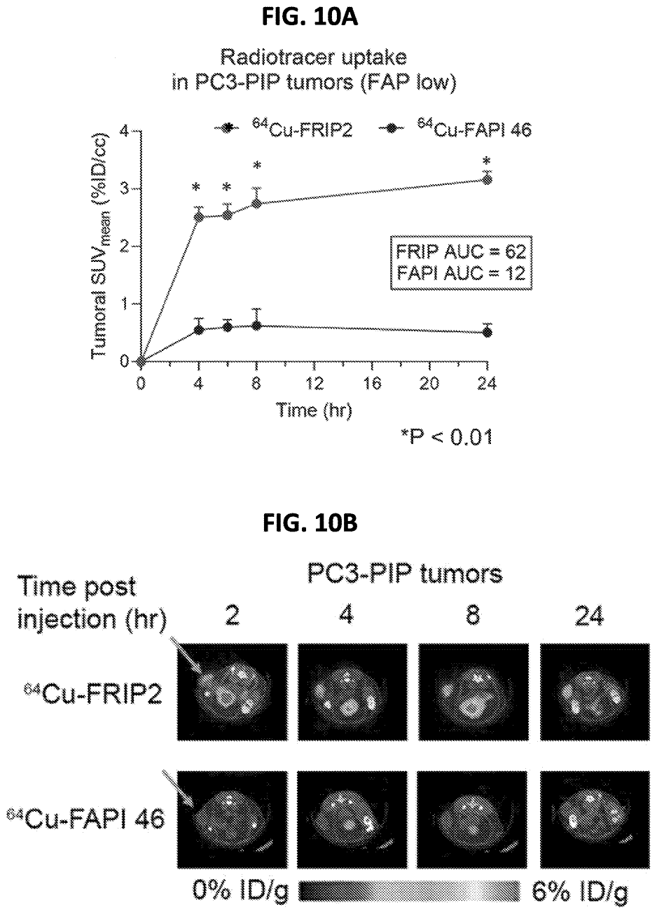

⁶⁴Cu-FRIP2 and ⁶⁴Cu-FAPI 46 were administered at equal doses and specific activities to mice bearing subcutaneous U87 tumors. PET studies (FIG. 9A) showed that tumoral uptake of ⁶⁴Cu-FRIP increased from 2-24 hours post injection and reached a SUVₘₑₐₙ of ˜9% ID/cc (FIG. 9B). In contrast, ⁶⁴Cu-FAPI 46 levels were significantly lower over the same time interval (SUVₘₑₐₙ ˜1% ID/cc). Notably, while the tumoral uptake of ⁶⁴Cu-FRIP2 was significantly higher than ⁶⁴Cu-FAPI 46, the clearance rate of each radiopharmaceutical was virtually identical. ⁶⁴Cu-FRIP2 and ⁶⁴Cu-FAPI 46 tumoral uptake was assessed in a separate cohort of mice bearing PC3 PIP xenografts, which have lower FAP compared to U87. PET data (FIG. 10A-10B) showed that ⁶⁴Cu-FRIP2 uptake in tumors was significantly higher than ⁶⁴Cu-FAPI 46. As observed in the U87 cohort, tumoral area under the curve for ⁶⁴Cu-FRIP2 was approximately 5 fold higher than ⁶⁴Cu-FAPI 46. The uptake of ⁶⁴Cu-FRIP2 was significantly lower in PC3 PIP tumors compared to U87 tumors, as expected.

| | Tumor dose (Gy/mCi) | | | | |
|---|---|---|---|---|---|
| | Cu-64 | Cu-67 | Y-90 | Lu-177 | Ac-225 |
| FRIP2 | 11 | 55 | 215 | 86 | 3981 |
| FAPI 46 | 2 | 11 | 42 | 16 | 720 |
| RIP/RLT ratio | 5.5 | 5.0 | 5.1 | 5.4 | 5.5 |

Estimated human absorbed doses for ⁶⁴Cu-FRIP2 based on a mouse dosimetry study are provided in the following table, where the values were calculated using OLINDA EXM software.

TABLE 1

| | Absorbed Dose (mGy/MBq) | |
|---|---|---|
| Organ | Adult Female (60 kg) | Adult Male (73 kg) |
| Adrenals | 0.0162 ± 0.004 | 0.0154 ± 0.002 |
| Brain | 0.0061 ± 0.001 | 0.0041 ± 0.001 |
| Breasts | 0.0117 ± 0.003 | 0.0119 ± 0.001 |
| Gall Bladder Wall | 0.0171 ± 0.003 | 0.0166 ± 0.002 |
| LLI Wall | 0.0144 ± 0.003 | 0.0151 ± 0.002 |
| Small Intestine | 0.0140 ± 0.003 | 0.0152 ± 0.002 |
| Stomach Wall | 0.0143 ± 0.003 | 0.0145 ± 0.002 |
| ULI Wall | 0.0148 ± 0.004 | 0.0151 ± 0.002 |
| Heart Wall | 0.0194 ± 0.002 | 0.0164 ± 0.001 |
| Kidneys | 0.0641 ± 0.018 | 0.0379 ± 0.011 |
| Liver | 0.0596 ± 0.018 | 0.0369 ± 0.009 |
| Lungs | 0.0220 ± 0.008 | 0.0156 ± 0.002 |
| Muscle | 0.0127 ± 0.003 | 0.0132 ± 0.002 |
| Ovaries | 0.0146 ± 0.003 | |
| Pancreas | 0.0159 ± 0.004 | 0.0156 ± 0.002 |
| Red Marrow | 0.0109 ± 0.002 | 0.0114 ± 0.001 |
| Osteogenic Cells | 0.0269 ± 0.006 | 0.0266 ± 0.003 |
| Skin | 0.0110 ± 0.002 | 0.0115 ± 0.001 |
| Spleen | 0.0142 ± 0.003 | 0.0142 ± 0.002 |
| Testes | | 0.0133 ± 0.002 |
| Thymus | 0.0132 ± 0.003 | 0.0133 ± 0.002 |
| Thyroid | 0.0121 ± 0.003 | 0.0131 ± 0.002 |
| Urinary Bladder Wall | 0.0703 ± 0.017 | 0.0702 ± 0.013 |
| Uterus | 0.0152 ± 0.003 | |
| Total Body | 0.0147 ± 0.003 | 0.0143 ± 0.001 |
| Effective Dose | 0.0198 ± 0.003 | 0.0176 ± 0.002 |
| (mSv/MBq) | | |

### Example 4—⁶⁷Cu-FRIP2 Potently Suppresses Tumor Growth

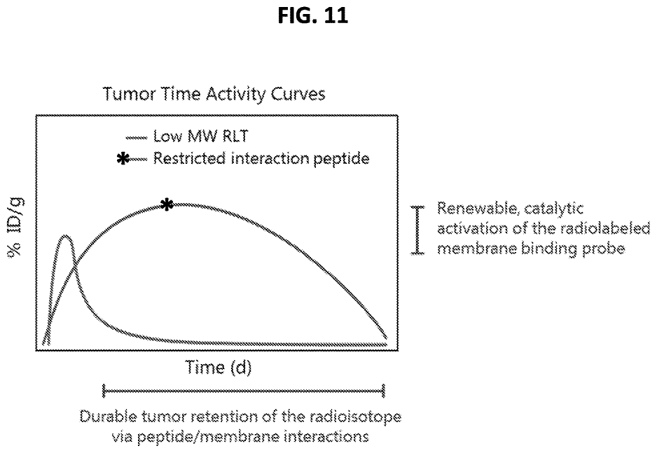

With the benefit of the data provided in the preceding Examples herein, it was expected that RIPs (including FRIPs) will be advantageous for targeted radiotherapy (TRT). In contrast to RLTs, RIPs accumulate in tumors through a renewable, catalytic mechanism, but also persist durably in tumors as cancer cells do not have efficient mechanisms to break up the peptide/membrane complex (FIG. 11). Provided in this example is proof of concept data employing a non-limiting example FRIP (⁶⁷Cu-FRIP2) demonstrating potent suppression of tumor growth by FRIPs.

Antitumor assessment studies were performed by treating mice bearing U87 MG tumors with ⁶⁷Cu-FRIP2. The antitumor assessment study was piloted with Cu-67 as it is beta emitter, isosteric with Cu-64, and this isotope is of clinical interest as demonstrated by a recent trial in the United States targeting SSTR2 positive neuroendocrine tumors with ⁶⁷Cu-SARTATE (NCT04023331). To synthesize ⁶⁷Cu-FRIP2, ⁶⁷CuCl₂ was coupled to DOTA-FRIP2 using the same protocol as applied for ⁶⁴Cu-FRIP2. The radiotracer was synthesized to >95% radiochemical yield and purity.

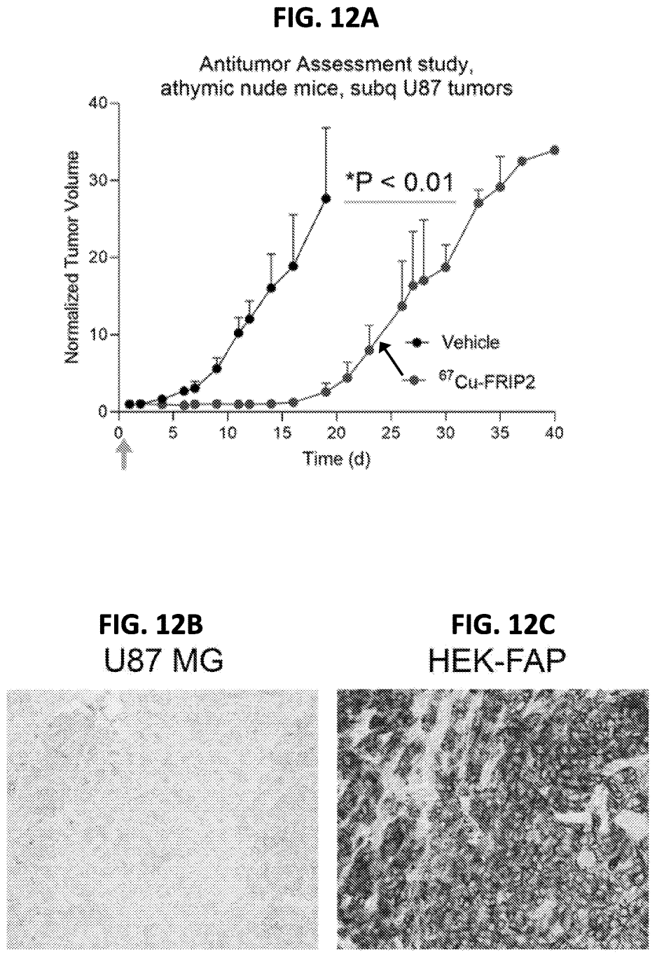

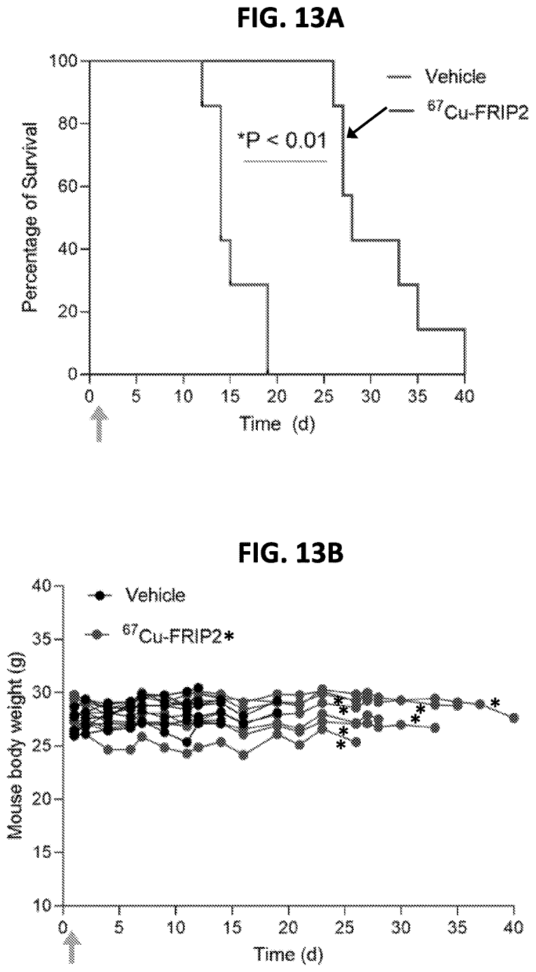

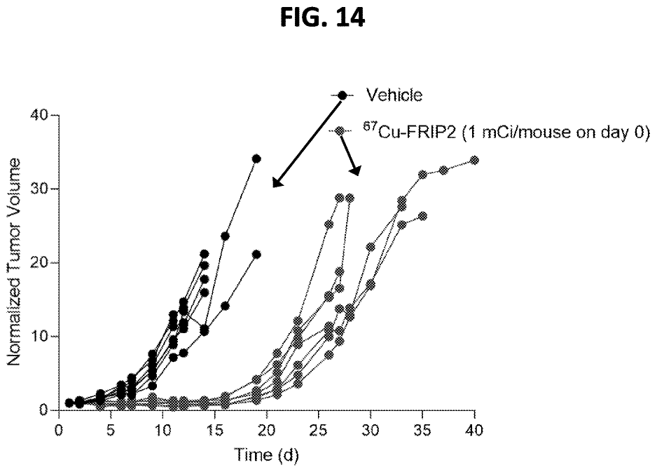

Male athymic nu/nu mice bearing subcutaneous U87 MG tumors received one IV dose of ⁶⁷Cu-FRIP2 (1 mCi/mouse) or vehicle (n=8 mice/arm). Data is shown in FIG. 12A-12C, FIG. 13A-13B and FIG. 14. The single dose of ⁶⁷Cu-FRIP2 significantly inhibited U87 MG tumor growth (FIG. 12A) and increased survival (FIG. 13A). Survival was benchmarked using the endpoints of (1) tumor volume exceeding 2000 mm³ or (2) weight loss exceeding 20%. Moreover, the treatment was well tolerated, as no significant loss in body mass was detected (FIG. 13B). A spider plot showing the individual tumor volume changes for the mice represented is shown in FIG. 14.

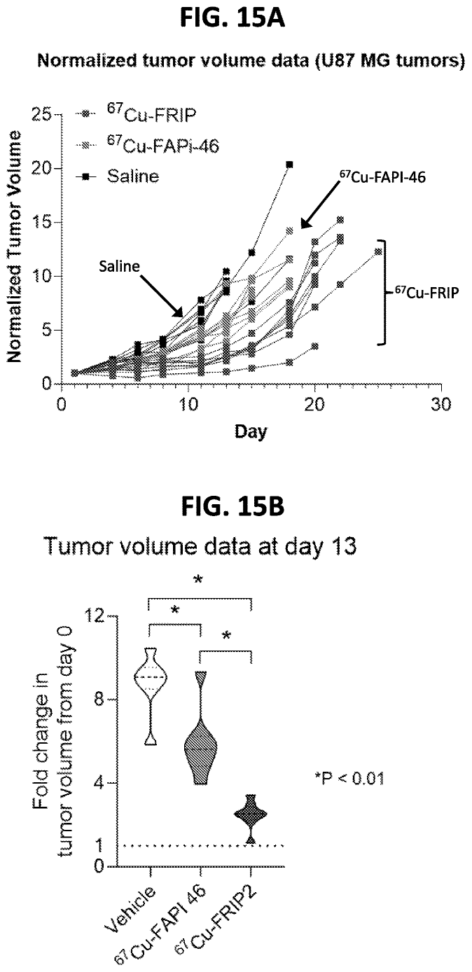

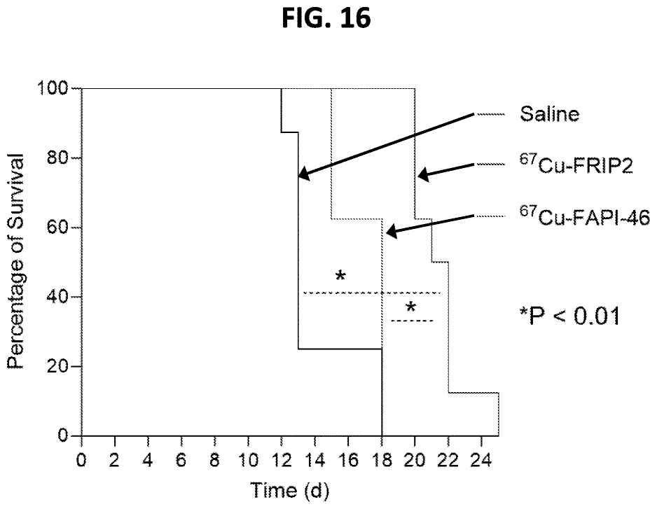

Tested next was whether ⁶⁷Cu-FRIP2 more potently suppressed tumor growth compared to ⁶⁷Cu-FAPI 46. Data is shown in FIG. 15A-15B and FIG. 16. Mice bearing subcutaneous U87 MG xenografts were treated with vehicle or a single IV dose of ⁶⁷Cu-FRIP2 (1 mCi/mouse) or ⁶⁷Cu-FAPI 46 (1 mCi/mouse). As shown, ⁶⁷Cu-FRIP2 more potently suppressed tumor growth compared to ⁶⁷Cu-FAPI 46 or vehicle.

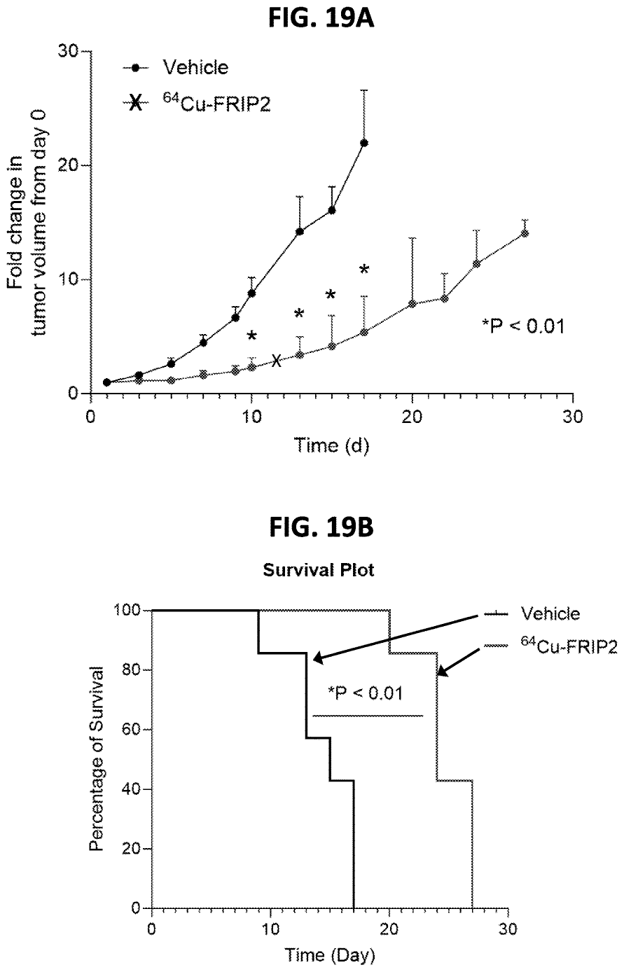

Assessed next was whether ⁶⁴Cu-FRIP2 administered at a high dose can suppress tumor growth. Male athymic nu/nu mice bearing subcutaneous U87 MG xenografts were treated with vehicle or IV dose of ⁶⁴Cu-FRIP2 (1.5 mCi/mouse) on day 0 and day 8 (n=8 mice/arm). ⁶⁷Cu-FRIP2 suppressed tumor growth compared to vehicle (FIG. 19A). Survival data plotted on a Kaplan-Meier curve showed significantly extended survival (P<0.01) of the treated arm compared to the vehicle arm (FIG. 19B).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A pro-peptide comprising:
a membrane interacting domain;
a masking domain that, when linked to the membrane interacting domain, is effective to inhibit interaction of the membrane interacting domain with a phospholipid bilayer; and
a fibroblast activation protein alpha (FAPα) cleavage site disposed between the membrane interacting domain and the masking domain.

2.-3. (canceled)

4. The pro-peptide of claim 1, wherein the membrane interacting domain comprises a protein from the Temporin family.

5. The pro-peptide of claim 1, wherein the membrane interacting domain comprises the amino acid sequence X^{a}X^{b}X^{c} X^{d}X^{e}X^{f}Y^{a}X^{g}X^{h}Y^{b}Y*XⁱX^{j}, wherein X^{a}, X^{b}, X^{c}, X^{d}, X^{e}, X^{f}, X^{g}, X^{h}, Xⁱ, and X^{j} are hydrophobic amino acid residues, Y^{a} and Y^{b} are hydrophilic amino acid residues, and Y* is a charged amino acid residue.

6. The pro-peptide of claim 5, wherein the membrane interacting domain comprises the amino acid sequence FVQWFSKFLGRIL (SEQ ID NO:2) or the amino acid sequence FVQWFSKFLGKLL (SEQ ID NO:3).

7. (canceled)

8. The pro-peptide of claim 1, wherein the masking domain comprises a peptide derived from protease-activated receptor-1 (PAR-1).

9. The pro-peptide of claim 8, wherein the masking domain comprises the amino acid sequence (X^{a})(X^{b})PND(X^{c})YEPF (SEQ ID NO:52), wherein X^{a} is R or Q, X^{b} is N or D, and X^{c} is K or Q.

10. The pro-peptide of claim 9, wherein the masking domain comprises the amino acid sequence QDPNDQYEPF (SEQ ID NO:35) or the amino acid sequence RNPNDKYEPF (SEQ ID NO:36).

11. (canceled)

12. The pro-peptide of claim 1, wherein the FAPα cleavage site comprises the FAPα specific motif GP in the P2-P1 positions.

13. The pro-peptide of claim 12, wherein the FAPα cleavage site comprises the amino acid sequence YHGPLAHX (SEQ ID NO:37), HIGPTAAY (SEQ ID NO:38), XXIPTNIR (SEQ ID NO:39), HQGPFWML (SEQ ID NO:40), XXGPKLTY (SEQ ID NO:41), HYGPTVNK (SEQ ID NO:42), XXWPMGMY (SEQ ID NO:43), XXFPNMWS (SEQ ID NO:44), XXGSQVFS (SEQ ID NO:45), XXMPEEVA (SEQ ID NO:46), or XXHPTKSF (SEQ ID NO:47), wherein X is any amino acid.

14.-15. (canceled)

16. The pro-peptide of claim 1, comprising from N-terminal to C-terminal: the membrane interacting domain, the FAPα cleavage site, and the masking domain.

17. The pro-peptide of claim 1, wherein the membrane interacting domain is conjugated to one or more therapeutic agents.

18. The pro-peptide of claim 17, wherein the one or more therapeutic agents comprise a cytotoxic agent, a toxin, a radiation-sensitizing agent, a radioisotope, or any combination thereof.

19. The pro-peptide of claim 18, wherein the one or more therapeutic agents comprise a radioisotope selected from the group consisting of: Actinium-225, Astatine-211, Bismuth-212, Bismuth-213, Bromine-76, Bromine-77, Calcium-47, Carbon-11, Carbon-14, Chromium-51, Cobalt-57, Cobalt-58, Copper-64, Erbium-169, Fluorine-18, Gallium-67, Gallium-68, Hydrogen-3, Indium-111, Iodine-123, Iodine-125, Iodine-131, Iron-59, Krypton-81m, Lead-212, Lutetium-177, Nitrogen-13, Oxygen-15, Phosphorus-32, Radium-223, Radium-224, Samarium-153, Selenium-75, Sodium-22, Sodium-24, Strontium-89, Technetium-99m, Thallium-201, Thorium-226, Thorium-227, Xenon-133, and Yttrium-9.

20.-21. (canceled)

22. The pro-peptide of claim 19, wherein the radioisotope is conjugated to the membrane interacting domain via a chelating moiety.

23. The pro-peptide of claim 22, wherein the chelating moiety comprises 1,4,7,10-tetraazacyclododecane-1,4,7,10-tetraacetic acid (DOTA).

24. (canceled)

25. A pharmaceutical composition, comprising:
the pro-peptide of claim 1; and
a pharmaceutically acceptable carrier.

26. A method of treating a condition associated with FAPα expression in a subject in need thereof, the method comprising administering an effective amount of the pro-peptide of claim 17 to the subject.

27.-40. (canceled)

41. A method of detectably labeling a phospholipid bilayer of a cell in the presence of FAPα activity, the method comprising:
contacting the pro-peptide of claim 1 with FAPα contributing to the FAPα activity, wherein:
the membrane interacting domain is detectably labeled, and
the FAPα cleavage site of the pro-peptide is cleaved by the FAPα to release a cleavage product comprising the detectably labeled membrane interacting domain, such that the detectably labeled membrane interacting domain interacts with the phospholipid bilayer of the cell and detectably labels the phospholipid bilayer of the cell in the presence of FAPα activity.

42. A method of assessing for FAPα activity in a subject, the method comprising:
administering the pro-peptide of claim 1 to the subject, wherein the membrane interacting domain is detectably labeled, wherein at sites of FAPα activity in the subject, the FAPα cleavage site of the pro-peptide is cleaved by FAPα to release cleavage products comprising the detectably labeled membrane interacting domain, and wherein the cleavage products interact with the phospholipid bilayers of cells at the sites of FAPα activity in the subject; and
assessing for the presence or absence of cells labeled with the cleavage products,
wherein the presence of cells labeled with the cleavage products indicates FAPα activity in a subject.

43. (canceled)


SEQUENCE LISTING (53 sequences)

SEQ ID NO: 1 - protein, 13 aa, unidentified
  VARIANT 4: note = I or L
  VARIANT 10: note = S or G
FLPXIASLLXKLL

SEQ ID NO: 2 - protein, 13 aa, unidentified
FVQWFSKFLGRIL

SEQ ID NO: 3 - protein, 13 aa, unidentified
FVQWFSKFLGKLL

SEQ ID NO: 4 - protein, 13 aa, unidentified
FLPLIGRVLSGIL

SEQ ID NO: 5 - protein, 13 aa, unidentified
LLPIVGNLLKSLL

SEQ ID NO: 6 - protein, 13 aa, unidentified
LLPILGNLLNGLL

SEQ ID NO: 7 - protein, 13 aa, unidentified
LLPIVGNLLNSLL

SEQ ID NO: 8 - protein, 13 aa, unidentified
VLPIIGNLLNSLL

SEQ ID NO: 9 - protein, 13 aa, unidentified
FLPLIGKVLSGIL

SEQ ID NO: 10 - protein, 13 aa, unidentified
FFPVIGRILNGIL

SEQ ID NO: 11 - protein, 10 aa, unidentified
LSPNLLKSLL

SEQ ID NO: 12 - protein, 10 aa, unidentified
LLPNLLKSLL

SEQ ID NO: 13 - protein, 13 aa, unidentified
FLPFLAKILTGVL

SEQ ID NO: 14 - protein, 13 aa, unidentified
FLPLFASLIGKLL

SEQ ID NO: 15 - protein, 13 aa, unidentified
FLPFLASLLTKVL

SEQ ID NO: 16 - protein, 13 aa, unidentified
FLPFLASLLSKVL

SEQ ID NO: 17 - protein, 13 aa, unidentified
FLPFLATLLSKVL

SEQ ID NO: 18 - protein, 14 aa, unidentified
SILPTIVSFLSKVF

SEQ ID NO: 19 - protein, 14 aa, unidentified
SILPTIVSFLSKFL

SEQ ID NO: 20 - protein, 14 aa, unidentified
SILPTIVSFLTKFL

SEQ ID NO: 21 - protein, 14 aa, unidentified
FILPLIASFLSKFL

SEQ ID NO: 22 - protein, 13 aa, unidentified
VLPLISMALGKLL

SEQ ID NO: 23 - protein, 14 aa, unidentified
NFLGTLINLAKKIM

SEQ ID NO: 24 - protein, 14 aa, unidentified
FLPILINLIHKGLL

SEQ ID NO: 25 - protein, 13 aa, unidentified
FLPIVGKLLSGLL

SEQ ID NO: 26 - protein, 12 aa, unidentified
FLPIASLLGKYL

SEQ ID NO: 27 - protein, 13 aa, unidentified
FISAIASMLGKFL

SEQ ID NO: 28 - protein, 13 aa, unidentified
FLSAIASMLGKFL

SEQ ID NO: 29 - protein, 13 aa, unidentified
FISAIASFLGKFL

SEQ ID NO: 30 - protein, 14 aa, unidentified
FLFPLITSFLSKVL

SEQ ID NO: 31 - protein, 13 aa, unidentified
FLPAIAGILSQLF

SEQ ID NO: 32 - protein, 13 aa, unidentified
FLPLIAGLLGKLF

SEQ ID NO: 33 - protein, 12 aa, unidentified
ILGTILGLLKGL

SEQ ID NO: 34 - protein, 14 aa, Rana japonica
FFPIGVFCKIFKTC

SEQ ID NO: 35 - protein, 10 aa, synthetic construct
QDPNDQYEPF

SEQ ID NO: 36 - protein, 10 aa, synthetic construct
RNPNDKYEPF

SEQ ID NO: 37 - protein, 8 aa, synthetic construct
YHGPLAHX

SEQ ID NO: 38 - protein, 8 aa, synthetic construct
HIGPTAAY

SEQ ID NO: 39 - protein, 8 aa, synthetic construct
XXIPTNIR

SEQ ID NO: 40 - protein, 8 aa, synthetic construct
HQGPFWML

SEQ ID NO: 41 - protein, 8 aa, synthetic construct
XXGPKLTY

SEQ ID NO: 42 - protein, 8 aa, synthetic construct
HYGPTVNK

SEQ ID NO: 43 - protein, 8 aa, synthetic construct
XXWPMGMY

SEQ ID NO: 44 - protein, 8 aa, synthetic construct
XXFPNMWS

SEQ ID NO: 45 - protein, 8 aa, synthetic construct
XXGSQVFS

SEQ ID NO: 46 - protein, 8 aa, synthetic construct
XXMPEEVA

SEQ ID NO: 47 - protein, 8 aa, synthetic construct
XXHPTKSF

SEQ ID NO: 48 - protein, 8 aa, synthetic construct
XSGPDYQK

SEQ ID NO: 49 - protein, 8 aa, synthetic construct
XXIARQPW

SEQ ID NO: 50 - protein, 8 aa, synthetic construct
GMGPFHIV

SEQ ID NO: 51 - protein, 11 aa, synthetic construct
FVQWFSKFLGK

SEQ ID NO: 52 - protein, 10 aa, synthetic construct
  VARIANT 1: note = R or Q
  VARIANT 2: note = N or D
  VARIANT 6: note = K or Q
XXPNDXYEPF

SEQ ID NO: 53 - protein, 29 aa, synthetic construct
FVQWFSKFLGKHQGPFWMLQDPNDQYEPF